(19) United States
(12) Patent Application Publication
(10) Pub. No.: US 2012/0064175 A1
(43) Pub. Date: Mar. 15, 2012

(54) HSP90 Inhibitors for Treating Non-Small Cell Lung Cancer in Wild-Type EGFR and/or KRAS Patients

(75) Inventors: Vojo Vukovic, Winchester, MA (US); Florentina Teofilovici, Wilmington, MA (US)

(73) Assignee: Synta Pharmaceuticals Corp.

(21) Appl. No.: 13/231,183

(22) Filed: Sep. 13, 2011

Related U.S. Application Data
(63) Continuation-in-part of application No. PCT/US2011/037285, filed on May 20, 2010.
(60) Provisional application No. 61/382,400, filed on Sep. 13, 2010.
(60) Provisional application No. 61/346,570, filed on May 20, 2010.

(51) Int. Cl. A61K 31/4196; A61K 33/24; A61P 35/00

(52) U.S. Cl. 424/649; 514/384; 514/383

(57) ABSTRACT

Provided is a method for treating non-small cell lung cancer with wild-type EGFR gene and/or KRAS gene by administering to a subject in need thereof, an effective amount of a triazolone compound according to the following formula:

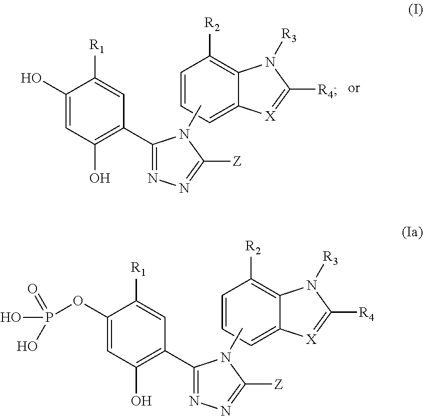

a tautomer, or a pharmaceutically acceptable salt thereof, wherein the variables in the structural formulae are defined herein.

### CROSS-REFERENCE TO RELATED PATENT APPLICATIONS

This application claims the benefit of U.S. Provisional Application No. 61/382,400, filed on Sep. 13, 2010. This application is a continuation-in-part of International Application No. PCT/US2011/37285, filed on May 20, 2011, which claims the benefit of U.S. Provisional Application No. 61/346,570, filed May 20, 2010. The entire teachings of all of the foregoing applications are incorporated herein by reference.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is now found that certain Hsp90 inhibitors are surprisingly effective at treating non-small cell lung cancer with wild-type EGFR and/or KRAS genes. The method of treating non-small cell lung cancer with wild-type EGFR gene and/or wild-type KRAS gene in a subject includes administering to said subject an effective amount of an Hsp90 inhibitor as described herein. In one embodiment, the method includes the steps of determining the status of the EGFR gene and/or KRAS gene of a subject with non-small cell lung cancer and administering an effective amount of an Hsp90 inhibitor as described herein wherein the presence of wild-type EGFR gene and/or wild-type KRAS gene in said subject is detected. In one embodiment, the method includes the steps of determining the status of the EGFR gene and/or KRAS gene of a subject with non-small cell lung cancer and administering to the subject an effective amount of an Hsp90 inhibitor described herein wherein the absence of mutated EGFR gene and/or mutated KRAS gene in said subject is detected.

The Hsp90 inhibitors suitable for the treatment include the triazolone compounds as described herein, geldanamycin derivatives, e.g., a benzoquinone or hygroquinone ansamycin such as IPI-493 (CAS No. 64202-81-9) or IPI-504 (CAS No. 857402-63-2); 17-AAG (CAS No. 75747-14-7), BIIB-021 (CNF-2024, CAS No. 848695-25-0), BIIB-028, AUY-922 (also known as VER-49009, CAS No. 747412-49-3), SNX-5422 (CAS No. 908115-27-5), AT-13387 (CAS No. 912999-49-6), XL-888, MPC-3100, CU-0305, 17-DMAG (CAS No. 467214-21-7), CNF-1010 (CAS No. 946090-39-7), Macbecin (e.g., Macbecin I (CAS No. 73341-72-7), Macbecin II (CAS No. 73341-73-8)), CCT-018159 (CAS No. 171009-07-7), CCT-129397 (CAS No. 940289-57-6), PU-H71 (CAS No. 873436-91-0), and PF-04928473 (SNX-2112, CAS No. 945626-71-1).

In one embodiment, the method includes treating non-small cell lung cancer in a mammal with wild-type EGFR (alternately, an “EGFR wild-type mammal”) or KRAS gene (alternately, a “KRAS wild-type mammal”) comprising administering to the mammal an effective amount of an Hsp90 inhibitor as described herein. In one embodiment, the Hsp90 inhibitor is Compound 1 as described herein.

In one embodiment, the method includes treating non-small cell lung cancer in a mammal with wild-type EGFR gene and wild-type KRAS gene (alternately, an “EGFR wild-type and KRAS wild-type mammal”) comprising administering to the mammal an effective amount of an Hsp90 inhibitor as described herein. In one embodiment, the Hsp90 inhibitor is Compound 1 as described herein.

In one embodiment, for methods described above, the non-small cell lung cancer is lung adenocarcinoma. In one embodiment, the type of lung adenocarcinoma is bronchioloalveolar carcinoma (BAC). In one embodiment, the BAC is non-mucinous. In another embodiment, the BAC is mucinous. In one embodiment, the non-small cell lung cancer is squamous cell lung carcinoma.

In one embodiment, for methods described above, the non-small cell lung cancer is Stage IIIB non-small cell lung cancer. In one embodiment, the non-small cell lung cancer is Stage IV non-small cell lung cancer.

In another embodiment, for methods described above, the method comprises administering to the mammal an effective amount of an additional anti-cancer agent. In one embodiment, the additional anti-cancer agent is paclitaxel. In one embodiment, the additional anti-cancer agent is docetaxel. In one embodiment, about 10 to about 50 mg/m², about 20 to about 40 mg/m², about 25 to about 35 mg/m², or about 30 mg/m² of docetaxel can be administered. In another embodiment, the method comprises administering to the mammal about 200 mg/m² of a compound described herein (e.g., Compound 1) and about 30 mg/m² of docetaxel once weekly. In one embodiment, the additional anti-cancer agent is cisplatin.

In another embodiment, for methods described above, the method comprises administering to the mammal between about 50 to about 500 mg/m², about 100 to about 300 mg/m², about 150 to 250 mg/m², about 175 to 275 mg/m², or about 200 mg/m² of a triazolone compound described herein (e.g., Compound 1) once weekly.

In one embodiment, the method includes the use of an Hsp90 inhibitor as described herein for the manufacture of a medicament for treating non-small cell lung cancer with wild-type EGFR and/or KRAS genes in a subject in need thereof. In another embodiment, the method includes the use of an Hsp90 inhibitor as described herein for the manufacture of a medicament for treating squamous cell lung carcinoma or lung adenocarcinoma in a subject in need thereof.

In one embodiment, the method includes the treatment of drug-resistant non-small cell lung cancer with wild-type EGFR gene and/or KRAS gene in a subject by administering to said subject an effective amount of an Hsp90 inhibitor as described herein. In one embodiment, the method of treatment of a drug-resistant non-small cell lung cancer may include the administration of one or more therapeutic agents in addition to an Hsp90 inhibitor as described herein.

### DETAILED DESCRIPTION OF THE INVENTION

### Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents. Examples of unsaturated alkyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkyl groups included in compounds described herein may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, cyclohexenyl, cyclooctenyl, cyclohexynyl, and the like. Cycloalkyl groups included in the compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be saturated or unsaturated, and may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker Alkoxy groups included in compounds described herein may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, an isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³². Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted. Each R³⁰, R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³⁰ or R³¹ or R³³ is optionally and independently unsubstituted. Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³, or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted. The variable k is 0, 1 or 2. In some embodiments, suitable substituents include C1-C4 alkyl, C1-C4 haloalkyl, C1-C4 alkoxy, C1-C4 haloalkoxy, C1-C4 hydroxyalkyl, halo, or hydroxyl.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In another embodiment, the subject is a human.

Unless indicated otherwise, the compounds described herein containing reactive functional groups, such as, for example, carboxy, hydroxy, thiol and amino moieties, also include corresponding protected derivatives thereof. “Protected derivatives” are those compounds in which a reactive site or sites are blocked with one or more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. GREENE, PROTECTING GROUPS IN ORGANIC SYNTHESIS, (John Wiley & Sons, Inc., 1981).

As used herein, the term “compound(s) described herein” or similar terms refers to a compound of formulae (I), or (Ia) or a compound in Tables 1 or 2 or a tautomer or pharmaceutically acceptable salt thereof. Also included in the scope of the embodiments are the neutral form of the compound or a solvate, clathrate, hydrate, polymorph, prodrug, or protected derivative of a compound of formulae (I), or (Ia), or a compound in Tables 1 or 2.

The compounds described herein may contain one or more chiral centers and/or double bonds and, therefore, exist as stereoisomers, such as double-bond isomers (i.e., geometric isomers), enantiomers or diastereomers. Each chemical structure shown herein, including the compounds described herein, encompass all of the corresponding compound' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds described herein are preferred.

When a disclosed compound is named or depicted by structure, it is to be understood that solvates (e.g., hydrates) of the compound or a pharmaceutically acceptable salt thereof is also included.

“Solvates” refer to crystalline forms wherein solvent molecules are incorporated into the crystal lattice during crystallization. Solvates may include water or nonaqueous solvents such as ethanol, isopropanol, DMSO, acetic acid, ethanolamine and ethyl acetate. When water is the solvent molecule incorporated into the crystal lattice of a solvate, it is typically referred to as a “hydrate”. Hydrates include stoichiometric hydrates as well as compositions containing variable amounts of water. When a compound is depicted by name or structure, it is to be understand that the anhydrous form of the compound is also included, i.e., the compound where solvent is substantially not incorporated within the crystalline structure.

When a disclosed compound is named or depicted by structure, it is to be understood that the compound, including solvates thereof, may exist in crystalline forms, non-crystalline forms or a mixture thereof. The compounds or solvates may also exhibit polymorphism (i.e., the capacity to occur in different crystalline forms). These different crystalline forms are typically known as “polymorphs.” It is to be understood that when named or depicted by structure, the disclosed compounds and solvates (e.g., hydrates) also include all polymorphs thereof. Polymorphs have the same chemical composition but differ in packing, geometrical arrangement and other descriptive properties of the crystalline solid state. Polymorphs, therefore, may have different physical properties such as shape, density, hardness, deformability, stability and dissolution properties. Polymorphs typically exhibit different melting points, IR spectra and X-ray powder diffraction patterns, which may be used for identification. One of ordinary skill in the art will appreciate that different polymorphs may be produced, for example, by changing or adjusting the conditions used in crystallizing the compound. For example, changes in temperature, pressure or solvent may result in different polymorphs. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salt, solvate or polymorph, are also included. “Clathrate” means a compound described herein, or a salt thereof, in the form of a crystal lattice that contains spaces (e.g., channels) that have a guest molecule trapped within (e.g., a solvent or water).

As used herein, and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated herein include analogs or derivatives of compounds of formulae (I) or (Ia) or a compound in Tables 1 or 2 that comprise biohydrolyzable moieties such as biohydrolyzable amides, biohydrolyzable esters, biohydrolyzable carbamates, biohydrolyzable carbonates, biohydrolyzable ureides and phosphate analogues. Prodrugs can typically be prepared using well-known methods, such as those described by BURGER'S MEDICINAL CHEMISTRY AND DRUG DISCOVERY, (Manfred E. Wolff Ed., 5^{th} ed. (1995)) 172-178, 949-982.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix.

The incidence of lung adenocarcinoma has been increasing in many developed Western nations in the past few decades, where it has become the most common major type of lung cancer in smokers and in lifelong nonsmokers. This cancer usually is seen peripherally in the lungs, as opposed to small cell lung cancer and squamous cell lung cancer, which both tend to be more centrally located, although it may also occur as central lesions. By unknown reasons, it often arises in relation to peripheral lung scars. Adenocarcinomas account for approximately 40% of lung cancers. Generally, adenocarcinomas grow more slowly and form smaller masses than the other subtypes. However, they tend to form metastases widely at an early stage. Adenocarcinoma is a non-small cell lung carcinoma, and as such, it is not as responsive to radiation therapy as is small cell lung carcinoma, but is rather treated by surgically. Adenocarcinomas are highly heterogeneous tumors, and several major histological subtypes are currently recognized: 1) a http://en.wikipedia.org/wiki/Adenocarcinoma_of_the_lung_cite_note-who2004-0#cite_note-who2004-0cinar adenocarcinoma; 2) papillary adenocarcinoma; 3) bronchioloalveolar adenocarcinoma; and 4) solid adenocarcinoma with mucin production.

As used herein, “BAC” refers to bronchioloalveolar carcinoma, a term describing certain variants of lung cancer arising in the distal bronchioles or alveoli that initially exhibit a specific non-invasive growth pattern. BAC is defined as a tumor that grows in a lepidic fashion along pre-existing airway structures, without detectable invasion or destruction of the underlying tissue, blood vessels, or lymphatics. Because invasion must be ruled out, BAC can be diagnosed only after complete sectioning and examination of the entire tumor, not using biopsy or cytology samples. BAC is considered a pre-invasive malignant lesion that, after further mutation and progression, eventually generates an invasive adenocarcinoma. BAC occurs in two major histopathological variants, mucinous BAC (m-BAC, 20%-25% of cases) and non mucinous BAC (nm-BAC, 75%-80% of cases). Non-mucinous BAC's are highly associated with classical EGFR mutations, and thus are often responsive to targeted chemotherapy with erlotinib and gefinitib. K-ras mutations are rare in nm-BAC. Mucinous BAC, in contrast, is much more highly associated with K-ras mutations and wild-type EGFR, and thus is usually insensitive to the EGFR tyrosine kinase inhibitors. Recent research has made it clear that nonmucinous and mucinous BACs are very different types of lung cancer. Mucinous BAC is much more likely to present with multiple unilateral tumors and/or in a unilateral or bilateral pneumonic form than nonmucinous BAC. The overall prognosis for patients with mucinous BAC is significantly worse than patients with nonmucinous BAC. (See Yousem S A, Beasley M B, Bronchioloalveolar carcinoma: a review of current concepts and evolving issues. Arch Pathol Lab Med 2007; 131:1027-32).

Her2 is a transmembrane tyrosine kinase cell surface growth factor receptor that is expressed in normal epithelial cells. Her2 has an extracellular domain that interacts with extracellular growth factors and an internal tyrosine kinase portion that transmits the external growth signal transduction pathways leading to cell growth and differentiation. Her2 is overexpressed in a significant proportion of malignancies, such as breast cancer, ovarian cancer, prostate cancer and gastric cancers, and is typically associated with a poor prognosis. It is encoded within the genome by HER2/neu, a known protooncogene. HER2 is thought to be an orphan receptor, with none of the EGF family of ligands able to activate it. However, ErbB receptors dimerise on ligand binding, and HER2 is the preferential dimerisation partner of other members of the ErbB family. The HER2 gene is a proto-oncogene located at the long arm of human chromosome 17(17q21-q22). HER2/neu (also known as ErbB-2) stands for “Human Epidermal growth factor Receptor 2” and is a protein giving higher aggressiveness in breast cancers. It is a member of the ErbB protein family, more commonly known as the epidermal growth factor receptor family. HER2/neu has also been designated as CD340 (cluster of differentiation 340) and p185. Approximately 15-20 percent of breast cancers have an amplification of the HER2/neu gene or overexpression of its protein product. Overexpression of this receptor in breast cancer is associated with increased disease recurrence and worse prognosis.

The Anaplastic Lymphoma Kinase (ALK) tyrosine kinase receptor is an enzyme that in humans is encoded by the ALK gene. The 2; 5 chromosomal translocation is frequently associated with anaplastic large cell lymphomas (ALCL5). The translocation creates a fusion gene consisting of the ALK (anaplastic lymphoma kinase) gene and the nucleophosmin (NPM) gene: the 3′ half of ALK, derived from chromosome 2, is fused to the 5′ portion of NPM from chromosome 5. The product of the NPM-ALK fusion gene is oncogenic. Other possible translocations of the ALK gene, such as the elm4 translocation, are also implicated in cancer.

B-Raf proto-oncogene serine/threonine-protein kinase (B-RAF), also known as V-raf murine sarcoma viral oncogene homolog B1, is a protein that in humans is encoded by the BRAF gene. The B-RAF protein is involved in sending signals in cells and in cell growth. The BRAF gene may be mutated, and the B-RAF protein altered, as an inherited mutation which causes birth defects, or as an acquired mutation (oncogene) in adults which causes cancer. Acquired mutations in this gene have also been found in cancers, including non-Hodgkin lymphoma, colorectal cancer, malignant melanoma, papillary thyroid carcinoma, non-small cell lung carcinoma, and adenocarcinoma of lung. More than 30 mutations of the BRAF gene associated with human cancers have been identified. The frequency of BRAF mutations varies widely in human cancers from more than 80% in melanomas, to as little as 0-18% in other tumors, such as 1-3% in lung cancers and 5% in colorectal cancer. In 90% of the cases, a Glu for Val substitution at residue 599 (now referred to as V600E) in the activation segment has been found in human cancers. This mutation has been widely observed in papillary thyroid carcinoma, colorectal cancer and melanomas. Depending on the type of mutation the kinase activity towards MEK may also vary. In the same paper it has been reported that most of the mutants stimulate enhanced B-RAF kinase activity toward MEK. However, a few mutants act through a different mechanism because although their activity toward MEK is reduced, they adopt a conformation that activates wild-type C-RAF, which then signals to ERK.

KRAS is a protein which in humans is encoded by the KRAS gene. Like other members of the Ras family, the KRAS protein is a GTPase and is an early player in many signal transduction pathways. KRAS is usually tethered to cell membranes because of the presence of an isoprenyl group on its C-terminus. When mutated, KRAS is an oncogene. The protein product of the normal KRAS gene performs an essential function in normal tissue signaling, and the mutation of a KRAS gene is an essential step in the development of many cancers. KRAS acts as a molecular on/off switch, and once it is turned on it recruits and activates proteins necessary for the propagation of growth factor and other receptors' signal, such as c-Raf and PI 3-kinase.

Phosphoinositide 3-kinases (PI 3-kinases or PI3Ks) are a family of enzymes involved in cellular functions such as cell growth, proliferation, differentiation, motility, survival and intracellular trafficking, which in turn are involved in cancer. PI3Ks are a family of related intracellular signal transducer enzymes capable of phosphorylating the 3 position hydroxyl group of the inositol ring of phosphatidylinositol (PtdIns). They are also known as phosphatidylinositol-3-kinases. The pathway, with oncogene PIK3CA and tumor suppressor PTEN (gene) is implicated in insensitivity of cancer tumors to insulin and IGF1, in calorie restriction. PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110α is mutated in many cancers. Many of these mutations cause the kinase to be more active. The PtdIns(3,4,5)P₃ phosphatase PTEN that antagonises PI 3-kinase signaling is absent from many tumours. Hence, PI 3-kinase activity contributes significantly to cellular transformation and the development of cancer.

AKT protein family, which members are also called protein kinases B (PKB) plays an important role in mammalian cellular signaling. Akt kinase is a serine/threonine kinase which is a downstream effector molecule of phosphoinositide 3-kinase and is involved in protecting a cell from apoptosis. Akt kinase is thought to be involved in the progression of cancer because it stimulates cell proliferation and suppresses apoptosis. Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Since it can block apoptosis, and thereby promote cell survival, Akt1 has been implicated as a major factor in many types of cancer. Akt is known to play a role in the cell cycle. Under various circumstances, activation of Akt was shown to overcome cell cycle arrest in G1 and G2 phases. Moreover, activated Akt may enable proliferation and survival of cells that have sustained a potentially mutagenic impact and, therefore, may contribute to acquisition of mutations in other genes.

Cdk4/cyclin D complexes are involved in phosphorylation of the retinoblastoma protein, which is an essential step in progression of a cell through the G1 phase of the cell cycle. Disruption of Hsp90 activity has been shown to decrease the half life of newly synthesized Cdk4.

Raf-1 is a MAP 3-kinase (MAP3K) which, when activated, can phosphorylate and activate the serine/threonine specific protein kinases ERK1 and ERK2. Activated ERKs play an important role in the control of gene expression involved in the cell division cycle, apoptosis, cell differentiation and cell migration.

The transforming protein of the Rous sarcoma virus, v-src, is a prototype of an oncogene family that induces cellular transformation (i.e., tumorogenesis) by non-regulated kinase activity. Hsp90 has been shown to complex with v-scr and inhibit its degradation.

p53 is a tumor suppressor protein that causes cell cycle arrest and apoptosis. Mutation of the p53 gene is found in about half of all human cancers, making it one of the most common genetic alterations found in cancerous cells. In addition, the p53 mutation is associated with a poor prognosis. Wild-type p53 has been shown to interact with Hsp90, but mutated p53 forms a more stable association with Hsp90 than wild-type p53 as a result of its misfolded conformation. A stronger interaction with Hsp90 protects the mutated protein from normal proteolytic degradation and prolongs its half-life. In a cell that is heterozygous for mutated and wild-type p53, inhibition of the stabilizing effect of Hsp90 causes mutant p53 to be degraded and restores the normal transcriptional activity of wild-type p53.

There are two classes of protein kinases (PKs): protein tyrosine kinases (PTKs), which catalyze the phosphorylation of tyrosine kinase residues, and the serine-threonine kinases (STKs), which catalyze the phosphorylation of serine or threonine residues. Growth factor receptors with PTK activity are known as receptor tyrosine kinases. Receptor tyrosine kinases are a family of tightly regulated enzymes, and the aberrant activation of various members of the family is one of the hallmarks of cancer. The receptor tyrosine kinase family can be divided into subgroups that have similar structural organization and sequence similarity within the kinase domain.

The members of the type III group of receptor tyrosine kinases include platelet-derived growth factor receptors (PDGF receptors alpha and beta), colony-stimulating factor receptor (CSF-1R, c-Fms), Fms-like tyrosine kinase (FLT3), and stem cell factor receptor (c-Kit). FLT3 is primarily expressed on immature hematopoietic progenitors and regulates their proliferation and survival.

The FLT3-ITD mutation is also present in about 3% of cases of adult myelodysplastic syndrome and some cases of acute lymphocytic leukemia (ALL). Advani, Current Pharmaceutical Design (2005), 11:3449-3457. FLT3 has been shown to be a client protein of Hsp90, and 17AAG, a benzoquinone ansamycin antibiotic that inhibits Hsp90 activity, has been shown to disrupt the association of FLT3 with Hsp90. The growth of leukemia cells that express either wild type FLT3 or FLT3-ITD mutations was found to be inhibited by treatment with 17AAG. Yao, et al., Clinical Cancer Research (2003), 9:4483-4493.

c-Kit is a membrane type III receptor protein tyrosine kinase which binds Stem Cell Factor (SCF) to its extraellular domain. c-Kit has tyrosine kinase activity and is required for normal hematopoiesis. However, mutations in c-Kit can result in ligand-independent tyrosine kinase activity, autophosphorylation and uncontrolled cell proliferation. Aberrant expression and/or activation of c-Kit has been implicated in a variety of pathologic states. For example, there is evidence of a contribution of c-Kit to neoplastic pathology, including its association with leukemias and mast cell tumors, small cell lung cancer, testicular cancer and some cancers of the gastrointestinal tract and central nervous system. In addition, c-Kit has been implicated in carcinogenesis of the female genital tract, sarcomas of neuroectodermal origin, and Schwann cell neoplasia associated with neurofibromatosis. Yang et al., J Clin Invest. (2003), 112:1851-1861; Viskochil, J Clin Invest. (2003), 112:1791-1793. c-Kit has been shown to be a client protein of Hsp90, and Hsp90 inhibitor 17AAG has been shown to induce apoptosis in Kasumi-1 cells, an acute myeloid leukemia cell line that harbors a mutation in c-Kit.

c-Met is a receptor tyrosine kinase that is encoded by the Met protooncogene and transduces the biological effects of hepatocyte growth factor (HGF), which is also referred to as scatter factor (SF). Jiang, et al., Crit. Rev. Oncol. Hemtol. (1999), 29: 209-248. c-Met and HGF are expressed in numerous tissues, although their expression is normally predominantly confined to cells of epithelial and mesenchymal origin, respectively. c-Met and HGF are required for normal mammalian development and have been shown to be important in cell migration, cell proliferation, cell survival, morphogenic differentiation and the organization of 3-dimensional tubular structures (e.g., renal tubular cells, gland formation, etc.). The c-Met receptor has been shown to be expressed in a number of human cancers. c-Met and its ligand, HGF, have also been shown to be co-expressed at elevated levels in a variety of human cancers, particularly sarcomas. However, because the receptor and ligand are usually expressed by different cell types, c-Met signaling is most commonly regulated by tumor-stroma (tumor-host) interactions. Furthermore, c-Met gene amplification, mutation and rearrangement have been observed in a subset of human cancers. Families with germine mutations that activate c-Met kinase are prone to multiple kidney tumors, as well as tumors in other tissues. Numerous studies have correlated the expression of c-Met and/or HGF/SF with the state of disease progression of different types of cancer, including lung, colon, breast, prostate, liver, pancreas, brain, kidney, ovarian, stomach, skin and bone cancers. Furthermore, the overexpression of c-Met or HGF have been shown to correlate with poor prognosis and disease outcome in a number of major human cancers including lung, liver, gastric and breast.

BCR-ABL is an oncoprotein with tyrosine kinase activity that has been associated with chronic myelogenous leukemia (CML), acute lymphocytic leukemia (ALL) in a subset of patients and acute myelogenous leukemia (AML) in a subset of patients. In fact, the BCR-ABL oncogene has been found in at least 90-95% of patients with CML, about 20% of adults with ALL, about 5% of children with ALL and in about 2% of adults with AML. The BCR-ABL oncoprotein is generated by the translocation of gene sequences from the c-ABL protein tyrosine kinase on chromosome 9 into the BCR sequences on chromosome 22, producing the Philadelphia chromosome. The BCR-ABL gene has been shown to produce at least three alternative chimeric proteins, p230 BCR-ABL, p210 BCR-ABL and p190 BCR-ABL, which have unregulated tyrosine kinase activity. The p210 BCR-ABL fusion protein is most often associated with CML, while the p190 BCR-ABL fusion protein is most often associated with ALL. BCR-ABL has also been associated with a variety of additional hematological malignancies including granulocytic hyperplasia, myelomonocytic leukemia, lymphomas and erythroid leukemia. BCR-ABL fusion proteins exist as complexes with Hsp90 and are rapidly degraded when the action of Hsp90 is inhibited. It has been shown that geldanamycin, a benzoquinone ansamycin antibiotic that disrupts the association of BCR-ABL with Hsp90, results in proteasomal degradation of BCR-ABL and induces apoptosis in BCR-ABL leukemia cells.

Epidermal Growth Factor Receptor (EGFR) is a member of the type 1 subgroup of receptor tyrosine kinase family of growth factor receptors which play critical roles in cellular growth, differentiation and survival. Activation of these receptors typically occurs via specific ligand binding which results in hetero- or homodimerization between receptor family members, with subsequent autophosphorylation of the tyrosine kinase domain. Specific ligands which bind to EGFR include epidermal growth factor (EGF), transforming growth factor α (TGFα), amphiregulin and some viral growth factors. Activation of EGFR triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation.

A number of human malignancies are associated with aberrant or overexpression of EGFR and/or overexpression of its specific ligands. Gullick, Br. Med. Bull. (1991), 47:87-98; Modijtahedi & Dean, Int. J. Oncol. (1994), 4:277-96; Salomon, et al., Crit. Rev. Oncol. Hematol. (1995), 19:183-232. Aberrant or overexpression of EGFR has been associated with an adverse prognosis in a number of human cancers, including head and neck, breast, colon, prostate, lung (e.g., NSCLC, adenocarcinoma and squamous lung cancer), ovarian, gastrointestinal cancers (gastric, colon, pancreatic), renal cell cancer, bladder cancer, glioma, gynecological carcinomas and prostate cancer. In some instances, overexpression of tumor EGFR has been correlated with both chemoresistance and a poor prognosis. Lei, et al., Anti-cancer Res. (1999), 19:221-28; Veale, et al., Br. J. Cancer (1993); 68:162-65. Mutations in EGFR are associated with many types of cancer as well. For example, EGFR mutations are highly prevalent in non-mucinous BAC patients. Finberg, et al., J. Mol. Diagnostics. (2007) 9(3):320-26.

As used herein, a “proliferative disorder” or a “hyperproliferative disorder,” and other equivalent terms, means a disease or medical condition involving pathological growth of cells. Proliferative disorders include cancer, smooth muscle cell proliferation, systemic sclerosis, cirrhosis of the liver, adult respiratory distress syndrome, idiopathic cardiomyopathy, lupus erythematosus, retinopathy, (e.g., diabetic retinopathy or other retinopathies), cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis and desmoid tumors. Non-cancerous proliferative disorders also include hyperproliferation of cells in the skin such as psoriasis and its varied clinical forms, Reiter's syndrome, pityriasis rubra pilaris, hyperproliferative variants of disorders of keratinization (e.g., actinic keratosis, senile keratosis), scleroderma, and the like. In one embodiment, the proliferative disorder is a myeloproliferative disorder. In one aspect, the myeloproliferative disorder is polycythemia vera, idiopathic myelofirbrosis, myelodysplastic syndrome, psoriasis or essential thrombocythemia. In one embodiment, the proliferative disorder expresses JAK2V617F mutation of JAK2. In an aspect of this embodiment, the proliferative disorder is polycythemia vera, idiopathic myelofirbrosis, or essential thrombocythemia. In one aspect, the proliferative disorder is polycythemia vera.

As used herein, the term “pharmaceutically acceptable salt” refers to a salt prepared from a compound of formulae (I) or (Ia) or a compound in Tables 1 or 2 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formulae (I) or (Ia) or a compound in Tables 1 or 2 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

As used herein, the term “pharmaceutically acceptable solvate,” is a solvate formed from the association of one or more pharmaceutically acceptable solvent molecules to one of the compounds of formulae (I) or (Ia) or a compound in Tables 1 or 2. The term “solvate” includes hydrates, e.g., hemihydrate, monohydrate, dihydrate, trihydrate, tetrahydrate, and the like.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound described herein being used. In cases where no amount is expressly noted, an effective amount should be assumed. Non-limiting examples of an effective amount of a compound described herein are provided herein below. In a specific embodiment, the method includes treating, managing, or ameliorating a disease or disorder, e.g. a proliferative disorder, or one or more symptoms thereof, comprising administering to a subject in need thereof a dose of the Hsp90 inhibitor at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month.

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer. In one embodiment, the Hsp90 inhibitor and the one or more additional therapeutic agents are dosed on independent schedules. In another embodiment, the Hsp90 inhibitor and the one or more additional therapeutic agents are dosed on approximately the same schedule. In another embodiment, the Hsp90 inhibitor and the one or more additional therapeutic agents are dosed concurrently or sequentially on the same day. In another embodiment, the Hsp90 inhibitor and the one or more additional therapeutic agents are dosed sequentially on different days.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a “racemic mixture” means about 50% of one enantiomer and about 50% of is corresponding enantiomer of the molecule. The combination encompasses all enantiomerically-pure, enantiomerically-enriched, diastereomerically pure, diastereomerically enriched, and racemic mixtures of the compounds described herein. Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds described herein are defined by their chemical structures and/or chemical names. Where a compound is referred to by both a chemical structure and a chemical name, and the chemical structure and the chemical name conflict, the chemical structure is determinative of the compound's identity.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

In one aspect, the method includes treating non-small cell lung cancer with wild-type EGFR gene and/or wild-type KRAS gene in a subject in need thereof, comprising administering to the subject an effective amount of a triazolone compound shown in Tables 1 or 2, or according to formula (I) or (Ia) as set forth below:

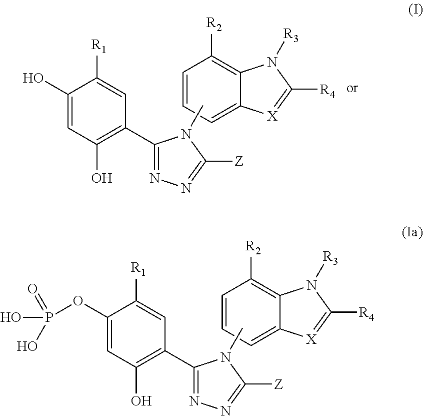

or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, (CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄.

In another embodiment, in formula (I) or (Ia), X is N.

In another embodiment, in formula (I) or (Ia), R₁ is selected from the group consisting of —H, lower alkyl, lower alkoxy, lower cycloalkyl, and lower cycloalkoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy.

In another embodiment, in formula (I) or (Ia), R₃ is selected from the group consisting of —H, a lower alkyl, a lower cycloalkyl, —C(O)N(R₂₇)₂, and —C(O)OH, wherein R₂₇ is —H or a lower alkyl.

In another embodiment, in formula (I) or (Ia), R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In one embodiment, R₄ is H or a lower alkyl.

In another embodiment, in formula (I) or (Ia), R₄ is selected from the group consisting of —H, methyl, ethyl, propyl, isopropyl or cyclopropyl.

In another embodiment, in formula (I) or (Ia), R₁ is selected from the group consisting of —H, —OH, —SH, —NH₂, a lower alkoxy and a lower alkyl amino.

In another embodiment, in formula (I) or (Ia), R₁ is selected from the group consisting of —H, —OH, methoxy and ethoxy.

In another embodiment, in formula (I) or (Ia), Z is —OH.

In another embodiment, in formula (I) or (Ia), Z is —SH.

In another embodiment, in formula (I) or (Ia), R₂ is selected from the group consisting of —H, —OH, —SH, —NH₂, a lower alkoxy and a lower alkyl amino.

In another embodiment, in formula (I) or (Ia), R₂ is selected from the group consisting of —H, —OH, methoxy, and ethoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is OH.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is SH.

In another embodiment, the triazolone compound is selected from the group consisting of:
3-(2,4-dihydroxy-5-ethyl-phenyl)-4-(1,3-dimethyl-indol-5-yl)-5-hydroxy-[1,2,4]triazole,
3-(2,4-dihydroxy-5-isopropyl-phenyl)-4-(1,3-dimethyl-indol-5-yl)-5-hydroxy-[1,2,4]triazole,
3-(2,4-dihydroxy-5-isopropyl-phenyl)-4-(1-methyl-indol-5-yl)-5-hydroxy-[1,2,4]triazole,
3-(2,4-dihydroxy-5-isopropyl-phenyl)-4-(1-isopropyl-indol-4-yl)-5-hydroxy-[1,2,4]triazole,
3-(2,4-dihydroxy-5-isopropyl-phenyl)-4-(1-methyl-indazol-5-yl)-5-mercapto-[1,2,4]triazole,
3-(2,4-dihydroxy-5-isopropyl-phenyl)-4-(1-methyl-indazol-6-yl)-5-mercapto-[1,2,4]triazole,
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole,
3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, the compound is selected from the group consisting of
3-(2,4-dihydroxy-5-ethyl-phenyl)-4-(1-ethyl-benzimidazol-4-yl)-5-mercapto-[1,2,4]triazole,
3-(2,4-dihydroxy-5-ethyl-phenyl)-4-(1-ethyl-benzimidazol-4-yl)-5-mercapto-[1,2,4]triazole HCL salt,
3-(2,4-dihydroxy-5-ethyl-phenyl)-4-(2-methyl-3-ethyl-benzimidazol-5-yl)-5-mercapto-[1,2,4]triazole,
3-(2,4-dihydroxy-5-ethyl-phenyl)-4-(1-ethyl-2-methyl-benzimidazol-5-yl)-5-mercapto-[1,2,4]triazole,
3-(2,4-dihydroxy-5-isopropyl-phenyl)-4-(1-methyl-2-trifluoromethyl-benzimidazol-5-yl)-5-mercapto-[1,2,4]triazole, or a tautomer, or a pharmaceutically acceptable salt thereof.

In another embodiment, the triazolone compound is selected from the group consisting of
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate,
4-(4-(1,3-dimethyl-1H-indol-5-yl)-5-hydroxy-4H-1,2,4-triazol-3-yl)-2-ethyl-5-hydroxyphenyl dihydrogen phosphate, or a tautomer, or a pharmaceutically acceptable salt thereof.

Hsp90 inhibitory compounds, as well as tautomers or pharmaceutically acceptable salts thereof. that may be used in the methods described herein are depicted in Tables 1 or 2.

TABLE 1

| | STRUCTURE | TAUTOMERIC STRUCTURE | NAME |
|---|---|---|---|
| 1 | 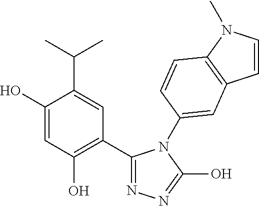 | 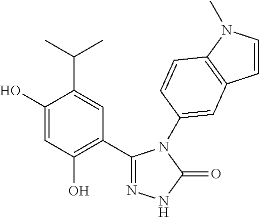 | 3-(2,4-DIHYDROXY-5- ISOPROPYL-PHENYL)-4-(1- METHYL-INDOL-5-YL)-5- HYDROXY-[1,2,4] TRIAZOLE |
| 2 | 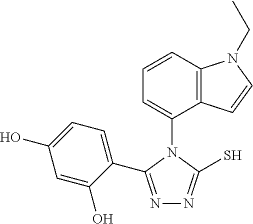 | 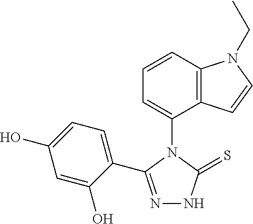 | 3-(2,4-DIHYDROXYPHENYL)-4- (1-ETHYL-INDOL-4-YL)-5- MERCAPTO-[1,2,4] TRIAZOLE |
| 3 | 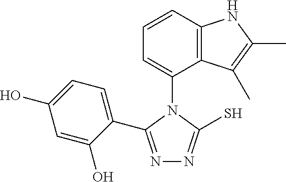 | 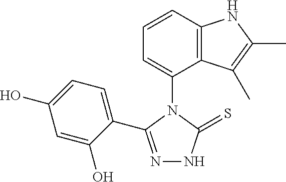 | 3-(2,4-DIHYDROXY-PHENYL)-4- (2,3-DIMETHYL-1H-INDOL-4- YL)-5-MERCAPTO-[1,2,4] TRIAZOLE |
| 4 | 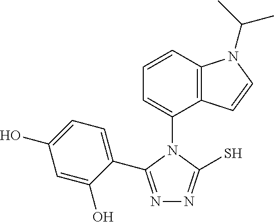 | 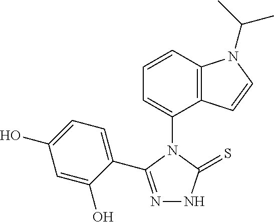 | 3-(2,4-DIHYDROXYPHENYL)-4- (1-ISOPROPYL-INDOL-4-YL)-5- MERCAPTO-[1,2,4] TRIAZOLE |
| 5 | 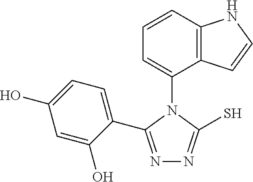 | 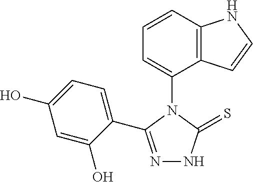 | 3-(2,4-DIHYDROXY-PHENYL)-4- (INDOL-4-YL)-5-MERCAPTO- [1,2,4] TRIAZOLE |
| 6 | 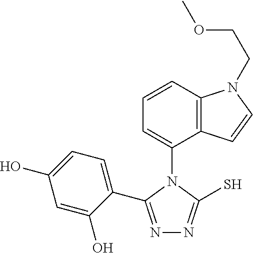 | 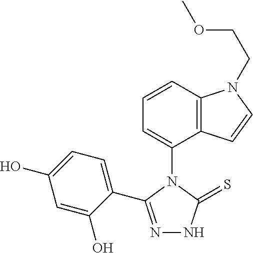 | 3-(2,4-DIHYDROXY-PHENYL)-4- [1-(2-METHOXYETHOXY)- INDOL- 4-YL]-5-MERCAPTO-[1,2,4] TRIAZOLE |
| 7 | 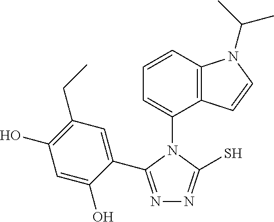 | 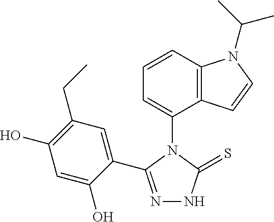 | 3-(2,4-DIHYDROXY-5-ETHYL- PHENYL)-4-(1-ISOPROPYL- INDOL-4-YL)-5-MERCAPTO- [1,2,4] TRIAZOLE |
| 8 | 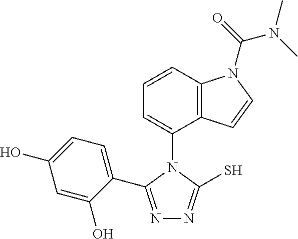 | 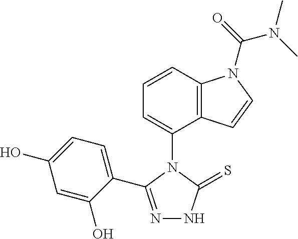 | 3-(2,4-DIHYDROXY-5-ETHYL- PHENYL)-4-[1-(DIMETHYL- CARBAMOYL)-INDOL-4-YL]-5- MERCAPTO-[1,2,4] TRIAZOLE |
| 9 | 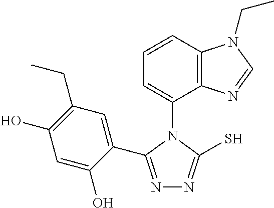 | 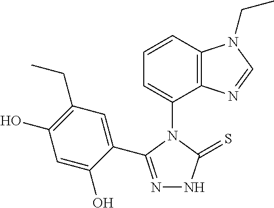 | 3-(2,4-DIHYDROXY-5-ETHYL- PHENYL)-4-(1-ETHYL- BENZOIMIDAZOL-4-YL)-5- MERCAPTO-[1,2,4] TRIAZOLE |
| 10 | 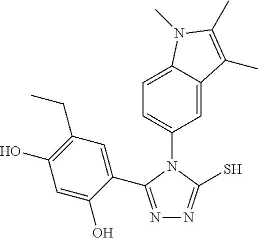 | 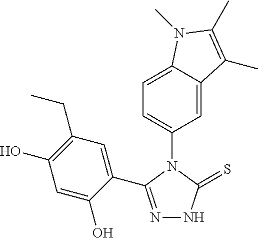 | 3-(2,4-DIHYDROXY-5-ETHYL- PHENYL)-4-(1,2,3-TRIMETHYL- INDOL-5-YL)-5-MERCAPTO- [1,2,4] TRIAZOLE |
| 11 | 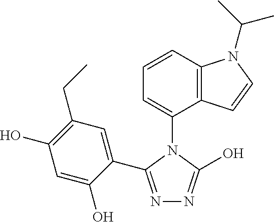 | 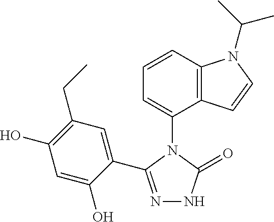 | 3-(2,4-DIHYDROXY-5-ETHYL- PHENYL)-4-(1-ISOPROPYL- INDOL-3-YL)-5- HYDROXY-[1,2,4] TRIAZOLE |
| 12 | 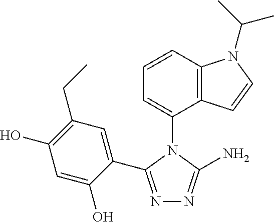 | 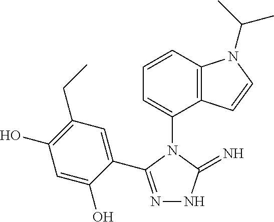 | 3-(2,4-DIHYDROXY-5-ETHYL- PHENYL)-4-(1-ISOPROPYL- INDOL-4-YL)-5-AMINO-[1,2,4] TRIAZOLE |
| 15 | 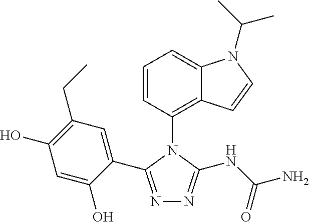 | | 3-(2,4-DIHYDROXY-5-ETHYL- PHENYL)-4-(1-ISOPROPYL- INDOL-4-YL)-5-UREIDO-[1,2,4] TRIAZOLE |
| 16 | 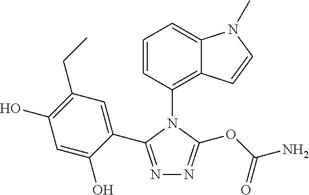 | | 3-(2,4-DIHYDROXY-5-ETHYL- PHENYL)-4-(1- METHYL-INDOL-4- YL)-5-CARBAMOYLOXY-[1,2,4] TRIAZOLE |
| 17 | 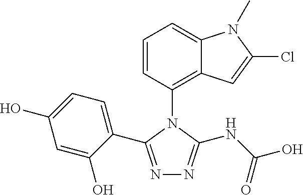 | | 3-(2,4-DIHYDROXY-PHENYL)-4- (1-METHYL-2- CHLORO-INDOL-4- YL)-5-CARBAMOYLOXY-[1,2,4] TRIAZOLE |
| 18 | 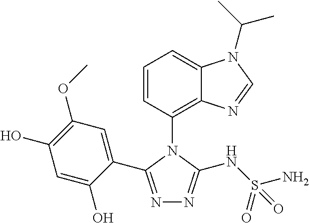 | | 3-(2,4-DIHYDROXY- 5-METHOXY- PHENYL)-4-(1-ISOPROPYL- BENZOIMIDAZOL-4-YL)-5- (SULFAMOYLAMINO)-[1,2,4] TRIAZOLE |
| 20 | 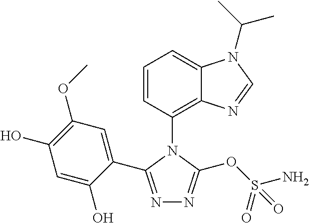 | | 3-(2,4-DIHYDROXY- 5-METHOXY- PHENYL)-4-(1-ISOPROPYL- BENZOIMIDAZOL-4-YL)-5- (SULFAMOYLOXY)-[1,2,4] TRIAZOLE |
| 21 | 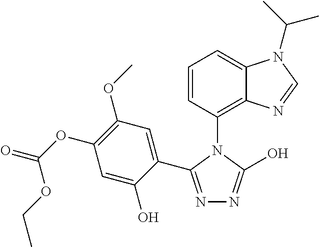 | 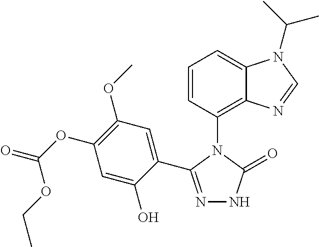 | 3-(2-HYDROXY-4- ETHOXYCARBONYOXY-5- METHOXY-PHENYL)-4-(1- ISOPROPYL- BENZOIMIDAZOL-4- YL)-5-HYDROXY-[1,2,4] TRIAZOLE |
| 22 | 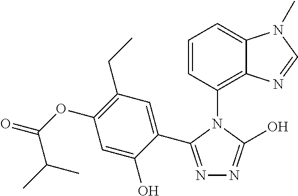 | 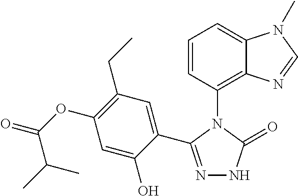 | 3-[2-HYDROXY-4- ISOBUTYRYLOXY-5-ETHYL- PHENYL]-4-(1-METHYL-BENZO- IMIDAZOL-4-YL)-5-HYDROXY- [1,2,4] TRIAZOLE |
| 23 | 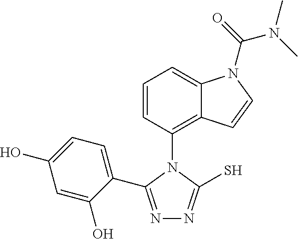 | 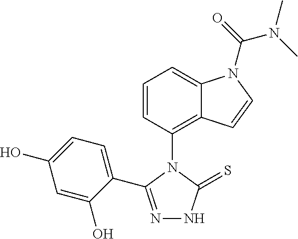 | 3-(2,4-DIHYDROXY-PHENYL)-4- (1-DIMETHYLCARBAMOYL- INDOL-4-YL)-5-MERCAPTO- [1,2,4] TRIAZOLE |
| 24 | 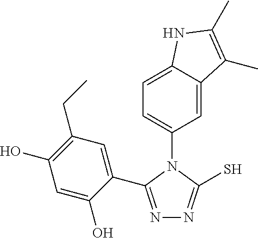 | 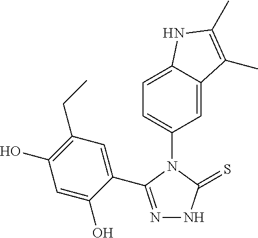 | 3-(2,4-DIHYDROXY-5-ETHYL- PHENYL)-4-(2,3-DIMETHYL- INDOL-5-YL)-5-MERCAPTO- [1,2,4] TRIAZOLE |
| 25 | 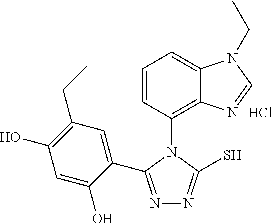 | 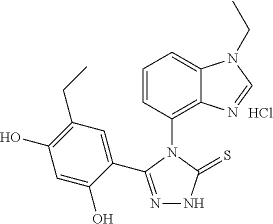 | 3-(2,4-DIHYDROXY-5-ETHYL- PHENYL)-4-(1-ETHYL-1H- BENZOIMIDAZOL-4-YL)-5- MERCAPTO-[1,2,4] TRIAZOLE, HCL SALT |
| 26 | 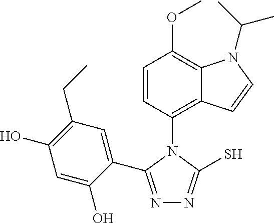 | 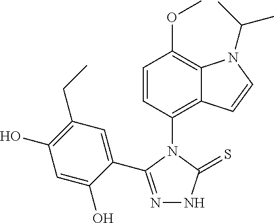 | 3-(2,4-DIHYDROXY-5-ETHYL- PHENYL)-4-(1-ISOPROPYL-7- METHOXY-INDOL-4-YL)-5- MERCAPTO-[1,2,4] TRIAZOLE |
| 27 | 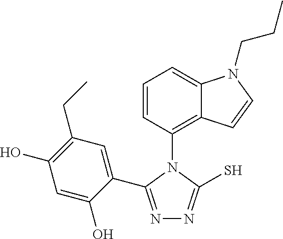 | 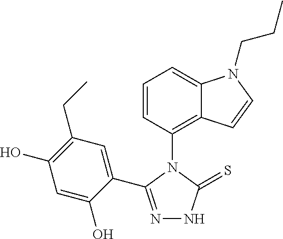 | 3-(2,4-DIHYDROXY-5-ETHYL- PHENYL)-4-(1- PROPYL-INDOL-4- YL)-5-MERCAPTO-[1,2,4] TRIAZOLE |
| 28 | 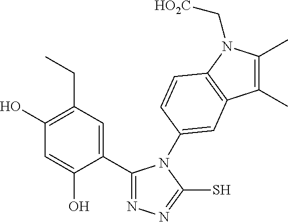 | 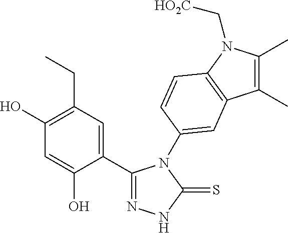 | 3-(2,4-DIHYDROXY-5-ETHYL- PHENYL)-4-(1-ACETYL-2,3- DIMETHYL-INDOL-5-YL)-5- MERCAPTO-[1,2,4] TRIAZOLE |
| 29 | 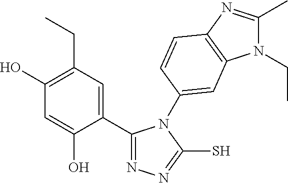 | 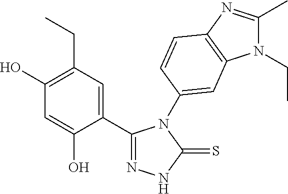 | 3-(2,4-DIHYDROXY-5-ETHYL- PHENYL)-4-(2-METHYL-3- ETHYL- BENZIMIDAZOL-5-YL)-5- MERCAPTO-[1,2,4] TRIAZOLE |
| 30 | 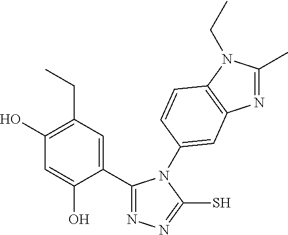 | 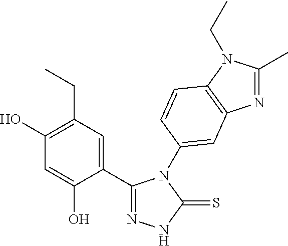 | 3-(2,4-DIHYDROXY-5-ETHYL- PHENYL)-4-(1-ETHYL-2- METHYL- BENZIMIDAZOL-5-YL)- 5-MERCAPTO-[1,2,4] TRIAZOLE |
| 31 | 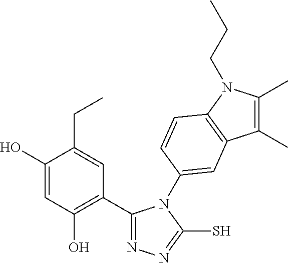 | 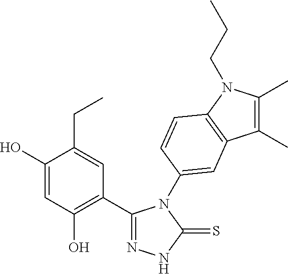 | 3-(2,4-DIHYDROXY-5-ETHYL- PHENYL)-4-(1-PROPYL-2,3- DIMETHYL-INDOL-5-YL)-5- MERCAPTO-[1,2,4] TRIAZOLE |
| 34 | 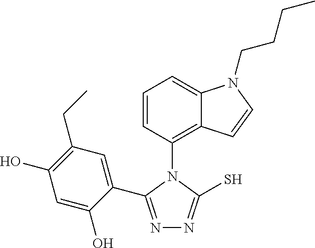 | 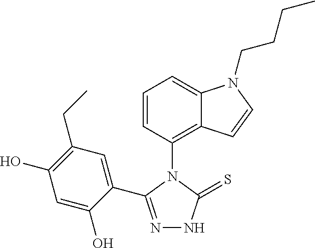 | 3-(2,4-DIHYDROXY-5-ETHYL- PHENYL)-4-(1- N-BUTYL-INDOL- 4-YL)-5-MERCAPTO-[1,2,4] TRIAZOLE |
| 35 | 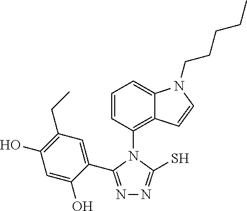 | 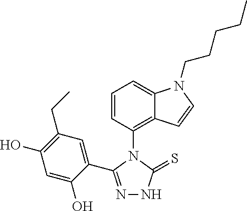 | 3-(2,4-DIHYDROXY-5-ETHYL- PHENYL)-4- (1-N-PENTYL-INDOL- 4-YL)-5-MERCAPTO-[1,2,4] TRIAZOLE |
| 36 | 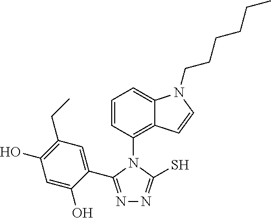 | 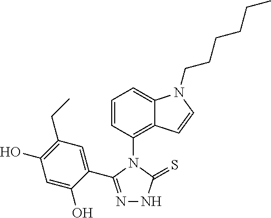 | 3-(2,4-DIHYDROXY-5-ETHYL- PHENYL)-4- (1-N-HEXYL-INDOL- 4-YL)-5-MERCAPTO-[1,2,4] TRIAZOLE |
| 37 | 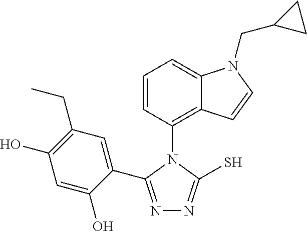 | 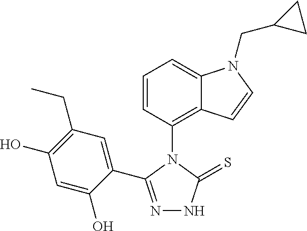 | 3-(2,4-DIHYDROXY-5- CYCLOPROPYL- PHENYL)-4-(1-(1- METHYLCYCLOPROPYL)- INDOL- 4-YL)-5-MERCAPTO-[1,2,4] TRIAZOLE |
| 38 | 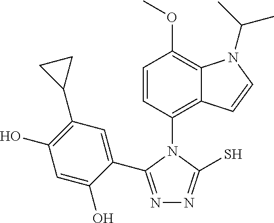 | 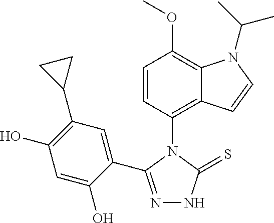 | 3-(2,4-DIHYDROXY-5- CYCLOPROPYL-PHENYL)-4-(1- 4-YL)-5-MERCAPTO-[1,2,4] TRIAZOLE |
| 39 | 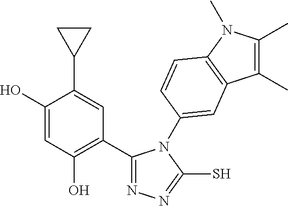 | 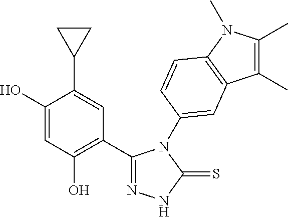 | 3-(2,4-DIHYDROXY-5- CYCLOPROPYL-PHENYL)-4- (1,2,3-TRIMETHYL- INDOL-5-YL)- 5-MERCAPTO-[1,2,4] TRIAZOLE |
| 40 | 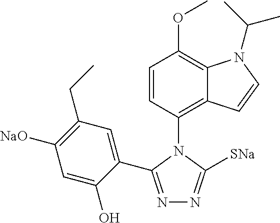 | | 3-(2,4-DIHYDROXY-5-ETHYL- PHENYL)-4-(1-ISOPROPYL-7- METHOXY-INDOL-4-YL)-5- MERCAPTO[1,2,4] TRIAZOLE DISODIUM SALT |
| 41 | 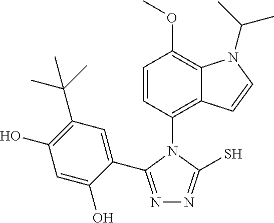 | 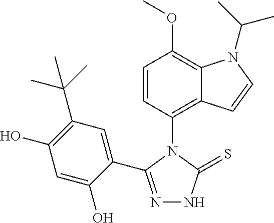 | 3-(2,4-DIHYDROXY-5-TERT- BUTYL-PHENYL)-4-(1- ISOPROPYL-7- METHYL-INDOL- 4-YL)-5-MERCAPTO-[1,2,4] TRIAZOLE |
| 42 | 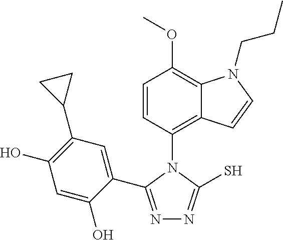 | 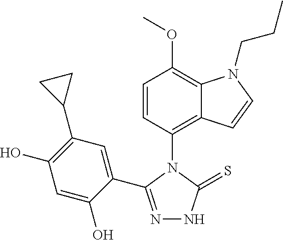 | 3-(2,4-DIHYDROXY-5- CYCLOPROPYL-PHENYL)-4-(1- PROPYL-7-METHOXY-INDOL-4- YL)-5-MERCAPTO-[1,2,4] TRIAZOLE |
| 43 | 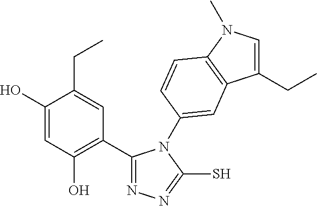 | 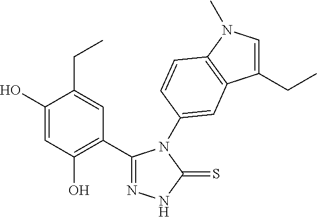 | 3-(2,4-DIHYDROXY-5-ETHYL- PHENYL)-4-(1-METHYL-3- ETHYL-INDOL-5-YL)-5- MERCAPTO-[1,2,4] TRIAZOLE |
| 44 | 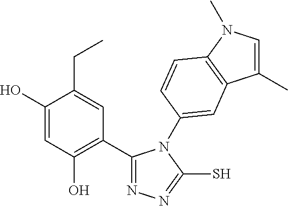 | 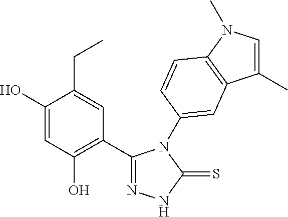 | 3-(2,4-DIHYDROXY-5-ETHYL- PHENYL)-4-(1,3-DIMETHYL INDOL-5-YL)-5-MERCAPTO- [1,2,4] TRIAZOLE |
| 45 | 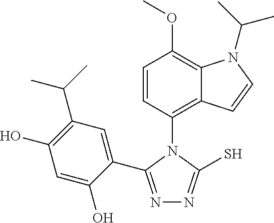 | 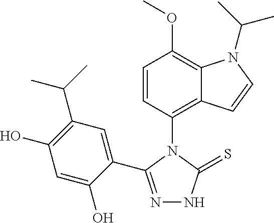 | 3-(2,4-DIHYDROXY-5- ISOPROPYL-PHENYL)-4-(1- ISOPROPYL-7- METHOXY-INDOL- 4-YL)-5-MERCAPTO-[1,2,4] TRIAZOLE |
| 46 | 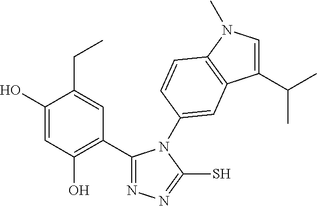 | 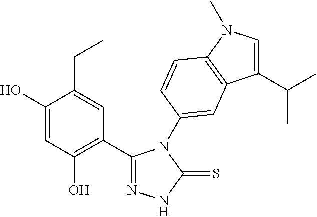 | 3-(2,4-DIHYDROXY-5-ETHYL- PHENYL)-4-(1-METHYL-3- ISOPROPYL-INDOL-5-YL)-5- MERCAPTO-[1,2,4] TRIAZOLE |
| 48 | 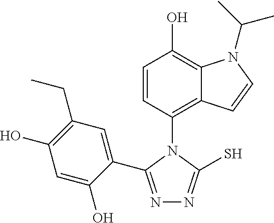 | 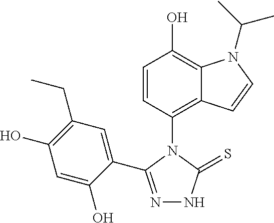 | 3-(2,4-DIHYDROXY-5-ETHYL- PHENYL)-4-(1-ISOPROPYL-7- HYDROXY-INDOL-4-YL)-5- MERCAPTO-[1,2,4] TRIAZOLE |
| 49 | 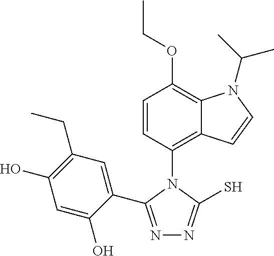 | 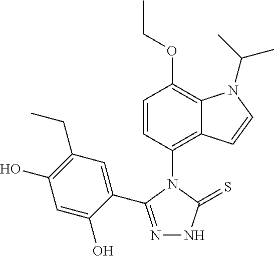 | 3-(2,4-DIHYDROXY-5-ETHYL- PHENYL)-4-(1-ISOPROPYL-7- ETHOXY-INDOL-4-YL)-5- MERCAPTO-[1,2,4] TRIAZOLE |
| 50 | 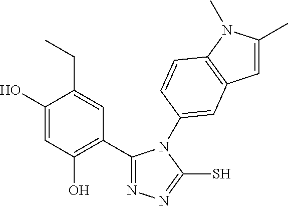 | 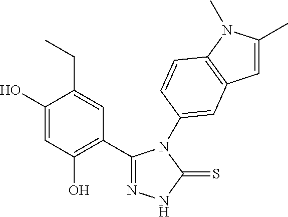 | 3-(2,4-DIHYDROXY-5-ETHYL- PHENYL)-4-(1,2-DIMETHYL- INDOL-5-YL)-5-MERCAPTO- [1,2,4] TRIAZOLE |
| 51 | 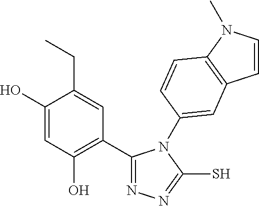 | 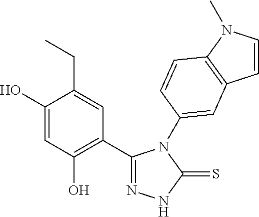 | 3-(2,4-DIHYDROXY-5-ETHYL- PHENYL)-4-(N-METHYL-INDOL- 5-YL)-5-MERCAPTO-[1,2,4] TRIAZOLE |
| 55 | 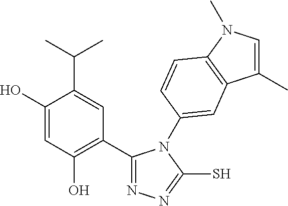 | 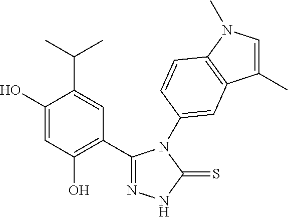 | 3-(2,4-DIHYDROXY-5- ISOPROPYL-PHENYL)-4-(1,3- DIMETHYL-INDOL-5-YL)-5- MERCAPTO-[1,2,4] TRIAZOLE |
| 56 | 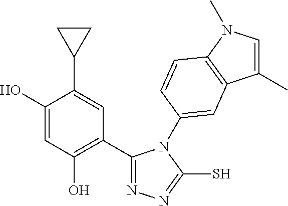 | 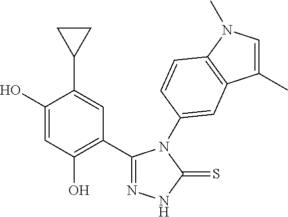 | 3-(2,4-DIHYDROXY-5- CYCLOPROPYL- PHENYL)-4-(1,3- DIMETHYL-INDOL-5-YL)-5- MERCAPTO-[1,2,4] TRIAZOLE |
| 57 | 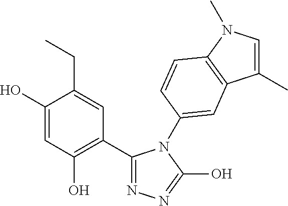 | 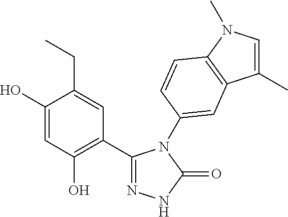 | 3-(2,4-DIHYDROXY-5-ETHYL- PHENYL)-4-(1,3-DIMETHYL- INDOL-5-YL)-5- HYDROXY-[1,2,4] TRIAZOLE |
| 58 | 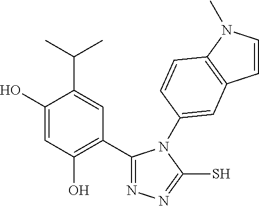 | 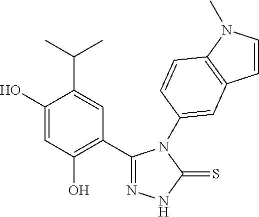 | 3-(2,4-DIHYDROXY-5- ISOPROPYL-PHENYL)-4-(N- METHYL-INDOL-5-YL)-5- MERCAPTO-[1,2,4] TRIAZOLE |
| 59 | 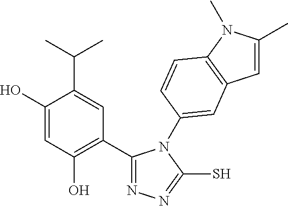 | 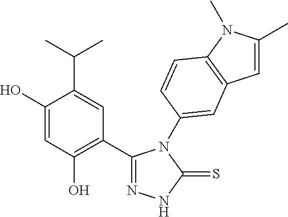 | 3-(2,4-DIHYDROXY-5- ISOPROPYL-PHENYL)-4-(1,2- DIMETHYL-INDOL-5-YL)-5- MERCAPTO-[1,2,4] TRIAZOLE |
| 60 | 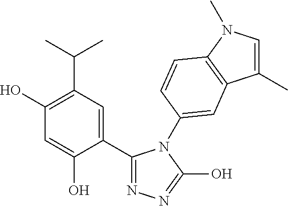 | 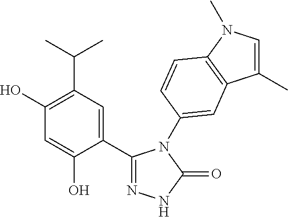 | 3-(2,4-DIHYDROXY-5- ISOPROPYL-PHENYL)-4-(1,3- DIMETHYL-INDOL-5-YL)-5- HYDROXY-[1,2,4] TRIAZOLE |
| 62 | 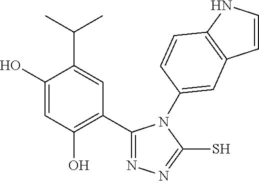 | 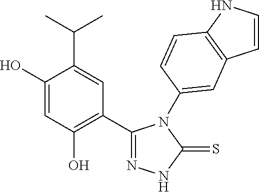 | 3-(2,4-DIHYDROXY-5- ISOPROPYL-PHENYL)-4-(1H- INDOL-5-YL)-5-MERCAPTO- [1,2,4] TRIAZOLE |
| 63 | 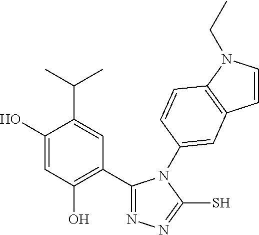 | 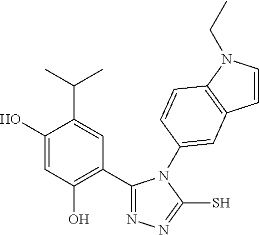 | 3-(2,4-DIHYDROXY-5- ISOPROPYL-PHENYL)-4-(1- ETHYL-INDOL-5-YL)-5- MERCAPTO-[1,2,4] TRIAZOLE |
| 64 | 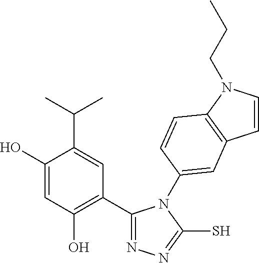 | 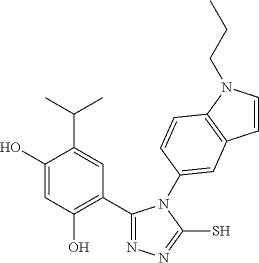 | 3-(2,4-DIHYDROXY-5- ISOPROPYL-PHENYL)-4-(1- PROPYL-INDOL-5-YL)-5- MERCAPTO-[1,2,4] TRIAZOLE |
| 65 | 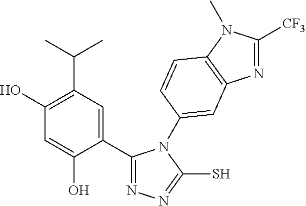 | 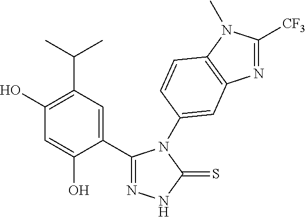 | 3-(2,4-DIHYDROXY-5- ISOPROPYL-PHENYL)-4-(1- METHYL-2- TRIFLUOROMETHYL- BENZIMIDAZOL-5-YL)-5- MERCAPTO-[1,2,4] TRIAZOLE |
| 66 | 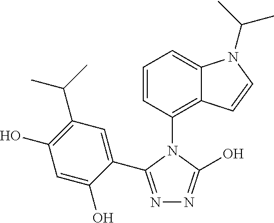 | 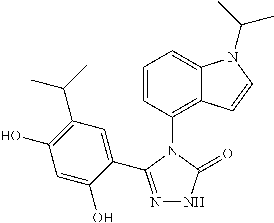 | 3-(2,4-DIHYDROXY-5- ISOPROPYL-PHENYL)-4-(1- ISOPROPYL-INDOL-4-YL)-5- HYDROXY-[1,2,4] TRIAZOLE |

TABLE 2
Compounds according to Formula (Ia)

| NO. | STRUCTURE | TAUTOMERIC STRUCTURE | NAME |
|---|---|---|---|
| 1A | 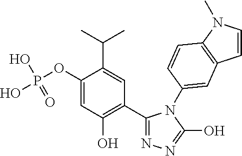 | 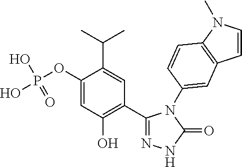 | 5-HYDROXY-4-(5- HYDROXY-4-(1-METHYL- 1H-INDOL-5-YL)-4H- 1,2,4-TRIAZOL-3-YL)-2- ISOPROPYLPHENYL DIHYDROGEN PHOSPHATE |
| 2A | 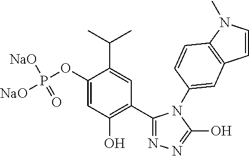 | 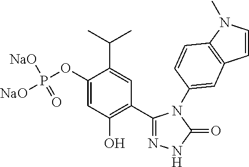 | SODIUM 5-HYDROXY-4- (5-HYDROXY-4-(1- METHYL-1H-INDOL-5- YL)-4H-1,2,4-TRIAZOL-3- YL)-2-ISOPROPYLPHENYL PHOSPHATE |
| 3A | 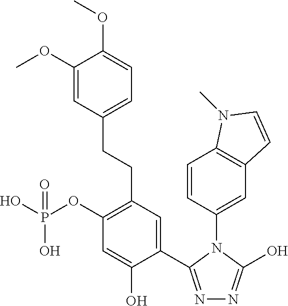 | 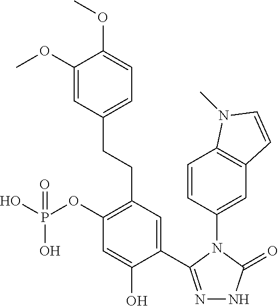 | 2-(3,4- DIMETHOXYPHENETHYL)- 5-HYDROXY-4-(5- HYDROXY-4-(1-METHYL- 1H-INDOL-5-YL)-4H- 1,2,4-TRIAZOL-3- YL)PHENYL DIHYDROGEN PHOSPHATE |
| 4A | 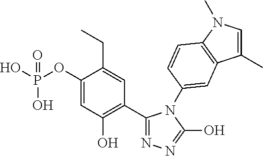 | 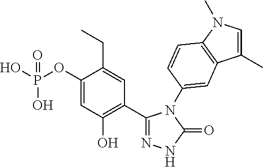 | 4-(4-(1,3-DIMETHYL-1H- INDOL-5-YL)-5- HYDROXY-4H-1,2,4- TRIAZOL-3-YL)-2-ETHYL- 5-HYDROXYPHENYL DIHYDROGEN PHOSPHATE |

The Hsp90 inhibitory compounds used in the disclosed methods can be prepared according to the procedures disclosed in U.S. Patent Publication No. 2006/0167070, and WO2009/023211.

These triazolone compounds typically can form a tautomeric structure as shown below and as exemplified by the tautomeric structures shown in Tables 1 and 2:

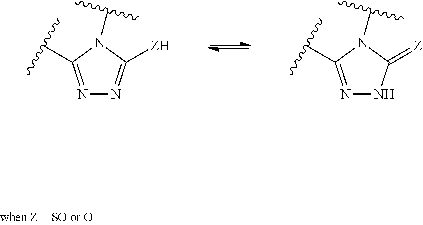

The method described herein includes treating non-small cell lung cancer with wild-type EGFR gene and/or wild-type KRAS gene in a subject in need thereof, comprising administering to the subject an Hsp90 inhibitor as described herein. In on embodiment, the Hsp90 inhibitor is a triazolone compound according to formulae (I) or (Ia) or a compound in Tables 1 or 2. In another embodiment, the method includes the steps of determining the status of the EGFR gene and/or KRAS gene of a subject with non-small cell lung cancer and administering an effective amount of an Hsp90 inhibitor according to formulae (I) or (Ia) or a compound in Tables 1 or 2 wherein the presence of wild-type EGFR gene and/or wild-type KRAS gene in said subject is detected. In one embodiment, the method includes the steps of determining the status of the EGFR gene and/or KRAS gene of a subject with non-small cell lung cancer and administering to the subject an effective amount of an Hsp90 inhibitor according to formulae (I) or (Ia) or a compound in Tables 1 or 2 wherein the absence of mutated EGFR gene and/or mutated KRAS gene in said subject is detected. In one embodiment, the Hsp90 inhibitor is Compound 1.

The determination of whether or not the EGFR gene and/or KRAS gene in a cell or sample from a subject is wild-type or mutated can be performed by various known biological methods such as, but not limited to, western blotting, ELISA, real-time PCR, immunohistochemistry, multi-analyte profiling beads, flow cytometry according to the procedures published and/or described herein.

The method further comprises administering one or more other therapies to the subject in need thereof (e.g., one or more therapeutic agents that are currently being used, have been used, are known to be useful or in development for use in the treatment or amelioration of cancer, or one or more symptoms associated with cancer).

In one embodiment, the one or more therapeutic agents described herein can be administered sequentially or concurrently. In certain embodiments, the one or more therapeutic agents described herein improve therapeutic effect of one or more compounds described herein by functioning together with the compounds to have an additive or synergistic effect. In certain embodiments, the one or more therapeutic agents described herein reduce the side effects associated with the therapies (e.g., therapeutic agents). In certain embodiments, the one or more therapeutic agents described herein reduce the effective dosage of one or more of the therapies.

The one or more therapeutic agents described herein can be administered to a subject, preferably a human subject, in the same pharmaceutical composition. In alternative embodiments, the one or more therapeutic agents described herein can be administered concurrently to a subject in separate pharmaceutical compositions. The therapeutic agents may be administered to a subject by the same or different routes of administration.

The therapeutic agents described herein can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

The method described herein also includes pharmaceutical formulations for the treatment, prophylaxis, and amelioration of non-small cell lung cancer. The pharmaceutical formulations described herein are formulated to be compatible with its intended route of administration. Examples of routes of administration include parenteral, e.g., intravenous, intradermal, subcutaneous, oral, intranasal (e.g., inhalation), transdermal (topical), transmucosal, and rectal administration. In a specific embodiment, the formulation is formulated in accordance with routine procedures as a pharmaceutical composition adapted for intravenous, subcutaneous, intramuscular, oral, intranasal or topical administration to human beings. In one embodiment, the formulation is formulated in accordance with routine procedures for subcutaneous administration to human beings.

The triazolone compounds described herein can be formulated into or administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

Other anti-proliferative or anti-cancer therapies may be combined with the compounds described herein to treat non-small cell lung cancer. Other therapies or anti-cancer agents that may be used in combination with the inventive anti-cancer agents described herein include surgery, radiotherapy (including gamma-radiation, neutron beam radiotherapy, electron beam radiotherapy, proton therapy, brachytherapy, and systemic radioactive isotopes), endocrine therapy, biologic response modifiers (including interferons, interleukins, and tumor necrosis factor (TNF)), hyperthermia and cryotherapy, agents to attenuate any adverse effects (e.g., antiemetics), and other approved chemotherapeutic drugs.

In one embodiment, the method of treating a subject with non-small cell lung cancer with wild-type EGFR gene or wild-type KRAS gene includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the triazolone compound is administered at an amount of about 200 mg/m². In one embodiment, the triazolone compound is administered at an amount of about 200 mg/m² once weekly. In one embodiment, the triazolone compound is administered at an amount of about 200 mg/m² twice weekly.

In one embodiment, the method includes the steps of determining the status of the EGFR gene and/or KRAS gene of a subject with non-small cell lung cancer and administering an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof wherein the presence of wild-type EGFR gene and/or wild-type KRAS gene in said subject is detected. In one embodiment, the triazolone compound is administered at an amount of about 200 mg/m². In another embodiment, the method includes the steps of determining the status of the EGFR gene and/or KRAS gene of a subject with non-small cell lung cancer and administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof wherein the absence of mutated EGFR gene and/or mutated KRAS gene in said subject is detected. In one embodiment, the triazolone compound is administered at an amount of about 200 mg/m².

In another embodiment, the method of treating a subject with non-small cell lung cancer with wild-type EGFR gene or wild-type KRAS gene includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art. Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising a triazolone compound described herein administered to prevent, treat, manage, or ameliorate cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 200 mg, 1 mg to 175 mg, 1 mg to 150 mg, 1 mg to 125 mg, 1 mg to 100 mg, 1 mg to 75 mg, 1 mg to 50 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 or 7 days, or once weekly, once every two weeks, once every three weeks or once monthly.

In certain embodiments, one or more compounds described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the method includes preventing, treating, managing, or ameliorating a proliferative disorders, such as cancer, or one or more symptoms thereof, comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

### EXAMPLES

### Example 1

### Synthesis of HSP90 Inhibitory Compounds

The triazolone Hsp90 inhibitory compounds used in the disclosed pharmaceutical compositions and methods herein can be prepared according to the procedures disclosed in U.S. Patent Publication No. 2006/0167070, and WO2009/023211.

### Example 2

### Compound 48 Displays Anti-tumor Activity Against Human Tumor Cells in a Nude Mouse Xenograft Model

The human squamous non-small cell lung cancer cell line, RERF-LC-AI (RCB0444; S. Kyoizumi, et al., Cancer. Res. 45:3274-3281, 1985), was obtained from the Riken Cell Bank (Tsukuba, Ibaraki, Japan). The cell line was cultured in growth media prepared from 50% Dulbecco's Modified Eagle Medium (high glucose), 50% RPMI Media 1640, 10% fetal bovine serum (FBS), 1% 100×L-glutamine, 1% 100× penicillin-streptomycin, 1% 100× sodium pyruvate and 1% 100×MEM non-essential amino acids. FBS was obtained from American Type Culture Collection (Manassas, Va., USA) and all other reagents were obtained from Invitrogen Corp. (Carlsbad, Calif., USA). Approximately 4-5×10(6) cells that had been cryopreserved in liquid nitrogen were rapidly thawed at 37° C. and transferred to a 175 cm² tissue culture flask containing 50 ml of growth media and then incubated at 37° C. in a 5% CO₂ incubator.

The growth media was replaced every 2-3 days until the flask became 90% confluent, typically in 5-7 days. To passage and expand the cell line, a 90% confluent flask was washed with 10 ml of room temperature phosphate buffered saline (PBS) and the cells were disassociated by adding 5 ml 1× trypsin-EDTA (Invitrogen) and incubating at 37° C. until the cells detached from the surface of the flask. To inactivate the trypsin, 5 ml of growth media was added and then the contents of the flask were centrifuged to pellet the cells. The supernatant was aspirated and the cell pellet was resuspended in 10 ml of growth media and the cell number determined using a hemocytometer. Approximately 1-3×10(6) cells per flask were seeded into 175 cm² flasks containing 50 ml of growth media and incubated at 37° C. in a 5% CO₂ incubator. When the flasks reached 90% confluence, the above passaging process was repeated until sufficient cells had been obtained for implantation into mice.

Seven to eight week old, female Crl:CD-1-nuBR (nude) mice were obtained from Charles River Laboratories (Wilmington, Mass., USA) Animals were housed 4-5/cage in micro-isolators, with a 12 hr/12 hr light/dark cycle, acclimated for at least 1 week prior to use and fed normal laboratory chow ad libitum. Studies were conducted on animals between 8 and 12 weeks of age at implantation. To implant RERF-LC-AI tumor cells into nude mice, the cells were trypsinized as above, washed in PBS and resuspended at a concentration of 50×10(6) cells/ml in 50% non-supplemented RPMI Media 1640 and 50% Matrigel Basement Membrane Matrix (#354234; BD Biosciences; Bedford, Mass., USA). Using a 27 gauge needle and 1 cc syringe, 0.1 ml of the cell suspension was injected subcutaneously into the flank of each nude mouse. Tumor volumes (V) were calculated by caliper measurement of the width (W), length (L) and thickness (T) of tumors using the following formula: V=0.5236×(L×W×T).

In vivo passaged RERF-LC-AI tumor cells (RERF-LC-AI^{IVP}) were isolated to improve the rate of tumor implantation relative to the parental cell line in nude mice. RERF-LC-AI tumors were permitted to develop in vivo until they reached approximately 250 mm³ in volume, which required approximately 3 weeks following implantation. Mice were euthanized via CO₂ asphyxiation and their exteriors sterilized with 70% ethanol in a laminar flow hood. Using sterile technique, tumors were excised and diced in 50 ml PBS using a scalpel blade. A single cell suspension was prepared using a 55 ml Wheaton Safe-Grind tissue grinder (catalog #62400-358; VWR International, West Chester, Pa., USA) by plunging the pestle up and down 4-5 times without twisting. The suspension was strained through a 70 μM nylon cell strainer and then centrifuged to pellet the cells. The resulting pellet was resuspended in 0.1 M NH₄Cl to lyse contaminating red blood cells and then immediately centrifuged to pellet the cells. The cell pellet was resuspended in growth media and seeded into 175 cm² flasks containing 50 ml of growth media at 1-3 tumors/flask or approximately 10×10(6) cells/flask. After overnight incubation at 37° C. in a 5% CO₂ incubator, non-adherent cells were removed by rinsing two times with PBS and then the cultures were fed with fresh growth media. When the flasks reached 90% confluence, the above passaging process was repeated until sufficient cells had been obtained for implantation into mice.

RERF-LC-AI^{IVP} cells were then implanted as above and tumors were permitted to develop in vivo until the majority reached an average of 100-200 mm³ in tumor volume, which typically required 2-3 weeks following implantation Animals with oblong or very small or large tumors were discarded, and only animals carrying tumors that displayed consistent growth rates were selected for studies Animals were randomized into treatment groups so that the average tumor volumes of each group were similar at the start of dosing.

The Hsp90 inhibitor, 17-allylamino-17-demethoxygeldanamycin (17-AAG), was employed as a positive control (Albany Molecular Research, Albany, N.Y., USA). Stock solutions of test articles were prepared by dissolving the appropriate amounts of each compound in dimethyl sulfoxide (DMSO) by sonication in an ultrasonic water bath. Stock solutions were prepared weekly, stored at −20° C. and diluted fresh each day for dosing. A solution of 20% Cremophor RH40 (polyoxyl 40 hydrogenated castor oil; BASF Corp., Aktiengesellschaft, Ludwigshafen, Germany) in 80% D5W (5% dextrose in water; Abbott Laboratories, North Chicago, Ill., USA) was also prepared by first heating 100% Cremophor RH40 at 50-60° C. until liquefied and clear, diluting 1:5 with 100% D5W, reheating again until clear and then mixing well. This solution was stored at room temperature for up to 3 months prior to use. To prepare formulations for daily dosing, DMSO stock solutions were diluted 1:10 with 20% Cremophor RH40. The final formulation for dosing contained 10% DMSO, 18% Cremophor RH40, 3.6% dextrose, 68.4% water and the appropriate amount of test article Animals were intraperitoneally (i.p.) injected with this solution at 10 ml per kg body weight on a schedule of 5 days per week (Monday, Tuesday, Wednesday, Thursday and Friday, with no dosing on Saturday and Sunday) for a total of 15 doses.

### Example 3

### A Non-Randomized, Open-label, Multi-Center, Multi-Cohort Phase 2 Study Evaluating the Efficacy and Safety of Compound 1 in Subjects with Stage IIIB or IV Non-Small Cell Lung Cancer

Patients with non-small cell lung cancer were enrolled in a Phase 2 clinical trial to evaluate the efficacy and safety of Compound 1. Various genotypic biomarkers were monitored for each patients, such as EGFR mutation, K-Ras mutation, and expression levels for EGFR and K-ras. Patients were divided into 4 cohorts, based on their EGFR and KRAS types. Cohort A included patients with EGFR mutations, who had received failed prior treatment with an approved EGFR TKi (erlotinib or gefitinib). Cohort B included patients with wild-type EGFR and K-ras mutations, who had received prior chemotherapy with at least 1 platinum doublet. Cohort C included patients with wild-type EGFR and wild-type K-ras, who had received prior chemotherapy with at least 1 platinum doublet. Cohort D includes patients with wild-type EGFR and wild-type KRAS with adenocarcinoma histology. The patients were treated with 200 mg/m² of Compound 1 once weekly by IV infusion for three consecutive weeks followed by a 1-week dose-free interval. Tumor assessments were performed at baseline and during the 1-week dose-free interval of every even cycle (e.g., Cycles 2, 4, 6 etc.). Patients were followed for survival every 4 weeks from the time of last dose of the test compound. The clinical data after 2 cycles of treatment are shown in the following table:

| | Best Response by RECIST* |
|---|---|
| Cohort A | 10 patients enrolled: 4 SD, 6 PD (1 ongoing) |
| EGFR mutation | |
| Cohort B | 4 patients enrolled: 3 PD, 1 other (0 ongoing) |
| KRAS mutation | |
| Cohort C | 15 patients enrolled: 1 PR, 10 SD, 2 PD, 2 |
| EGFR wt/KRAS wt | other (5 ongoing) |
| Cohort D | 35 patients enrolled: ongoing |
| EGFR wt and KRAS | |
| wt with adenocarcinoma | |
| histology | |

*CR = Complete Response, PR = Partial Response; SD = Stable Disease; PD = Progressive Disease

As shown in the above table, the compound has a 73% (11/15) DCR (disease control rate: percentage of patients achieving CR/PD/SD), higher than the DCR of 35-57% observed for Tarceva, Taxotere, Alimta and Nexavar. In addition, the compound was well tolerated at the 200 mg/m² once-weekly schedule, without the serious hepatic or ocular toxicities observed with other Hsp90 inhibitors, which is consistent with the Phase 1 results.

The phase 2 clinical trial was expanded from up to 69 patients to up to 146 patients based on the encouraging activity observed in the first stage of the two stage clinical trial described above. An additional cohort was created to allow certain patients to receive treatment with both Compound 1 and docetaxel. Patients in this cohort were treated with 1-hour infusion of 200 mg/m² of Compound 1 followed by a 1-hour infusion of 30 mg/m² of docetaxol once-weekly for three consecutive weeks followed by a 1-week dose-free interval. The data shows synergistic activity of docetaxel and Compound 1.

A Phase 2 clinical trial of Compound 1 in patients with Stage IIIB and Stage IV non-small cell lung cancer (NSCLC) was expanded from up to 69 patients to up to 146 patients based on encouraging activity observed in the first stage of the two stage clinical trial. Compound 1 is a potent, second-generation, small-molecule Hsp90 inhibitor, with a chemical structure unrelated to the first-generation, ansamycin family of Hsp90 inhibitors (e.g., 17-AAG or IPI-504).

The NSCLC trial was enrolling patients into cohorts defined by the mutational status of key genes, EGFR and KRAS, in order to identify cancer types especially responsive to Compound 1. In the first stage of this trial, patients with EGFR wild type and KRAS wild type, representing over 70% of all NSCL cancers, have shown a high disease control rate, over 70%. This early signal is very encouraging, particularly as the patients in this trial have been heavily pretreated and are refractory to many standard-of-care drugs for NSCLC. Also encouraging is that Compound 1 continues to be well tolerated at the 200 mg/m² once-weekly schedule, without the serious hepatic or ocular toxicities observed with other Hsp90 inhibitors, consistent with our Phase 1 results. Based on these findings, the investigators modified the protocol, and expanded the cohort in order to confirm and further characterize the observed activity.

An additional cohort was created to allow certain patients to receive treatment with both Compound 1 and docetaxel. Clinical and preclinical results provide a strong rationale for combining taxanes and Hsp90 inhibitors, with the potential for synergistic activity.

### About the Phase 2 Trial

The Phase 2 trial was initially designed to enroll up to 23 patients (14 in Stage 1, 9 in Stage 2) in each of three cohorts specified by cancer genetic profile. The cohorts are: EGFR mutation, KRAS mutation, and absence of EGFR and KRAS mutations (“wild type”). The recent amendment allows for two new cohorts. The first is an expansion cohort of up to 35 patients with EGFR and KRAS wild type. An additional up to 14 patients is allowed in this cohort for each of three additional disease profiles hypothesized to have enhanced sensitivity to Hsp90 inhibition. The second is a combination therapy cohort that allows certain patients from this trial to receive both docetaxel and Compound 1.

### Disease Control Rate in NSCLC

Disease control rate (DCR) consists of complete response (CR) plus partial response (PR) and stable disease (SD). According to recent studies, DCR at week 8 is a more powerful predictor of subsequent survival than is the traditional tumor response rate in advanced NSCLC, and provides an early assessment of subsequent outcome'.

Lung cancer is the leading cause of cancer-related mortality in the United States. Adenocarcinoma patients make up approximately 45% of the 222,250 new cases of NSCLC diagnosed in the United States each year², with approximately half of those patients having both EGFR and KRAS wild type gene mutations (wt-wt)³.

The five-year relative survival rate for NSCLC varies from 16% for patients diagnosed with regional metastatic stage disease to 2% for patients diagnosed with distant metastatic stage disease. (Source and further information: American Cancer Society, http://www.cancer.org.)

### REFERENCES

1. Lara, Prima N. et al, Disease Control Rate at 8 Weeks Predicts Clinical Benefit in Advanced Non-Small-Cell Lung Cancer: Results From Southwest Oncology Group Randomized Trials, JCO, Vol 26, 3, Jan. 20, 2008, pp 463-467
2. American Cancer Society website, accessed Sep. 8, 2010.
3. Soh, AACR 2010 Abstr 790, Mutations and copy number gains of EGFR and KRAS genes in lung adenocarcinomas.

### Example 4

### Once Weekly Administration of Compound 1

### Study Protocol:

This was an open-label Phase 1 dose-escalation study in subjects with solid tumors. The subject received 150 mg/m² of compound 1 during a 1-hour infusion 1 time per week for three consecutive weeks followed by a 1 week dose-free interval. Each four week period of treatment is considered one cycle. The subjects in this study had histologically- or cytologically-confirmed non-hematological malignancy that was metastatic or unresectable. The subjects were documented to be refractory to, or were not candidates for, current standard therapy. Subjects were assessed for response rate (CR, PR, SD) based on the Response Evaluation Criteria in Solid Tumors (RECIST). Durability of response was also measured.

This subject entered the study with stage IV mucinous BAC, with target lesions in the subcarinal lymph node and the right paratracheal lymph node. Additional lesions were in the mediastinal lymph nodes and diffuse lung lesions. The patient was diagnosed with BAC in 2006. Molecular profiling showed wild type KRAS and EGFR, and EGFR was not amplified on FISH testing. This subject stayed on study for thirteen complete cycles. Response and progression were evaluated in this study using the international criteria proposed by the RECIST Committee. Changes in only the largest diameter (unidimensional measurement) of the tumor lesions were used in the RECIST criteria. The RECIST criteria specify:

Complete Response (CR): Disappearance of all target lesions
Partial Response (PR): At least a 30% decrease in the sum of the longest diameter (LD) of target lesions, taking as reference the baseline sum LD.
Progressive Disease (PD): At least a 20% increase in the sum of the LD of target lesions, taking as reference the smallest sum LD recorded since the treatment started or the appearance of one or more new lesions.
Stable Disease (SD): Neither sufficient shrinkage to qualify for PR nor sufficient increase to qualify for PD, taking as reference the smallest sum LD since the treatment started. Stable disease was measured from the start of the treatment until the criteria for progression were met, taking as reference the smallest measurements recorded since the treatment started.

The best overall response was the best response recorded from the start of the treatment until disease progression/recurrence (taking as reference for progressive disease the smallest measurements recorded since the treatment started). The subject's best response assignment would depend on the achievement of both measurement and confirmation criteria.

### Past Chemotherapy Treatments:

| Previous Medications | Duration of Treatment | | Best Response |
|---|---|---|---|
| Erlotinib | 131 | days | PD |
| Erlotinib + | 65 | days | PD |
| Bevacizumab | | | |
| Carboplatin + | 107 | days | PD |
| Paclitaxel + | | | |
| Bevacizumab | | | |
| Pemetrexed | 49 | days | PD |
| Bortezomib + | 74 | days | PD |
| Toptecan | | | |
| Antineoplastic Agents | ~121 | days | SD |

Other Treatments: Surgery—once in 2004, twice in 2005
Treatment with Compound 1:
Dosage: 150 mg/m², once a week

| | Tumor Size | % Change | |
|---|---|---|---|
| Treatment Cycle | (SLD) | from Baseline | Response |
| 0 - baseline scan | 34 mm | — | — |
| 2 | 31 mm | −8.8% | SD |
| 4 | 25 mm | −26.5% | SD |
| 6 | 25 mm | −26.5% | SD |
| 8 | 25 mm | −26.5% | SD |
| 10 | 28 mm | −17.6% | SD |
| 12 | 25 mm | −26.5% | SD |

Number of cycles initiated: 13
Overall duration of stable disease: 344 days
Reason for discontinuing study: death from pneumonia (not treatment related)

As shown above, this was the seventh chemotherapeutic treatment that the subject tried. During treatment, the subject had diarrhea (grade 1) that was associated with treatment and also experienced some intermittent shortness of breath (grade 1) that was potentially associated with treatment with compound 1.

### Example 5

### Twice Weekly Administration of Compound 1

### Study Protocol:

This was an open-label Phase 1 dose-escalation study in subjects with solid tumors. The subject received 14 mg/m² of compound 1 during a 1-hour infusion 2 times per week for three consecutive weeks followed by a 1 week dose-free interval. Each four week period of treatment is considered one cycle. The subjects in this study had histologically- or cytologically-confirmed non-hematological malignancy that was metastatic or unresectable. The subjects were documented to be refractory to, or were not candidates for, current standard therapy.

This subject entered the study with stage IV BAC, with target lesions in the mediastinal lymph node and the right hepatic lobe. The subject also had multiple attenuated lesions in the liver. This subject stayed on study for three complete cycles. Response and progression were evaluated in this study using the international criteria proposed by the RECIST Committee. Durability of response was also measured.

Changes in only the largest diameter (unidimensional measurement) of the tumor lesions are used in the RECIST criteria described in Example 3.

### Past Chemotherapy Treatments:

| Previous medications | Duration of treatment | Best Response |
|---|---|---|
| Carboplatin + | 71 days | Unknown |
| Paclitaxel | | |
| Cetuximab | 113 days | PD |
| Pemetrexed | 492 days | SD |
| Erlotinib + | 17 days | PD |
| Tetracycline | | |
| Erlotinib | 129 days | PD |
| Gemcitabine | 56 days | PD |

Other Treatments: Surgery—once in 2006, once in 2008
Treatment with Compound 1:
Dosage: 14 mg/m², twice a week

| | Tumor Size | % Change | |
|---|---|---|---|
| Treatment Cycle | (SLD) | from Baseline | Response |
| 0 - baseline scan | 41 mm | — | — |
| 2 | 41 mm | 0% | SD |
| 3 | 43 mm | 4.9% | SD |

Number of cycles initiated: 3
Overall duration of stable disease: 93 days
Reason for discontinuing study: symptomatic deterioration

As detailed above, this was the seventh chemotherapeutic treatment that the subject tried. During treatment, the subject had some adverse events that were potentially associated with treatment, including weight loss, elevated aspartate aminotransferase levels (grade 1), and fatigue (grade 2 and 3).

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

1. A method of treating non-small cell lung cancer with wild-type EGFR gene or wild-type KRAS gene in a subject in need thereof, comprising administering to the subject an effective amount of a triazolone compound according to the following formulae: 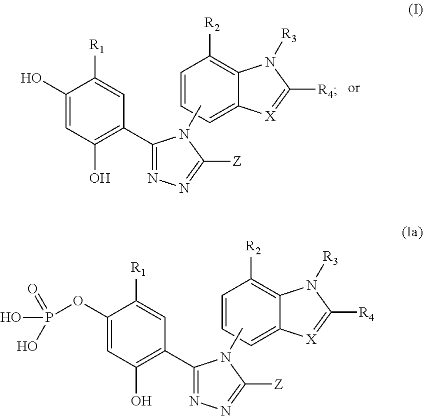
or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SR(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

2. A method of treating non-small cell lung cancer with wild-type EGFR gene and wild-type KRAS gene in a subject in need thereof, comprising administering to the subject an effective amount of a triazolone compound as defined in claim 1.

3. A method of treating non-small cell lung cancer with wild-type EGFR gene or wild-type KRAS gene in a subject in need thereof, comprising the steps of:
a) determining the status of the EGFR gene or KRAS gene in a sample from the subject; and
b) administering to the subject an effective amount of a triazolone compound as defined in claim 1 wherein the absence of the mutated EGFR gene or mutated KRAS gene is indicated.

4. A method of treating non-small cell lung cancer with wild-type EGFR gene and wild-type KRAS gene in a subject in need thereof, comprising the steps of:
a) determining the status of the EGFR gene and KRAS gene in a sample from the subject; and
b) administering to the subject an effective amount of a triazolone compound as defined in claim 1 wherein the absence of the mutated EGFR gene and mutated KRAS gene is indicated.

5. A method of treating non-small cell lung cancer in a subject in need thereof, comprising the steps of:
a) determining the status of the EGFR gene and/or KRAS gene in a sample from the non small cell lung cancer from the subject;
b) administering to the subject an effective amount of a triazolone compound as defined in claim 1, if the status of the EGFR and/or KRAS gene is wild type; or
c) administering to the subject an effective amount of an anti-cancer agent effective in the treatment of non-small cell lung cancer other than a triazolone compound as defined in claim 1, if the status of the EGFR and/or KRAS gene is mutated.

6. The method according to claim 1, wherein the triazolone compound is selected from the group consisting of:
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole, and
3-(2,4-dihydroxy-5-isopropyl-phenyl)-4-(1-propyl-indol-5-yl)-5-mercapto-[1,2,4]triazole,
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate, and
4-(4-(1,3-dimethyl-1H-indol-5-yl)-5-hydroxy-4H-1,2,4-triazol-3-yl)-2-ethyl-5-hydroxyphenyl dihydrogen phosphate,
or a tautomer, or a pharmaceutically acceptable salt thereof.

7. The method according to claim 1, wherein the triazolone compound is selected from the group consisting of:
3-(2,4-dihydroxy-5-ethyl-phenyl)-4-(1-isopropyl-7-methoxy-indol-4-yl)-5-mercapto-[1,2,4]triazole;
3-(2,4-dihydroxy-5-isopropyl-phenyl)-4-(N-methyl-indol-5-yl)-5-mercapto-[1,2,4]triazole;
3-(2,4-dihydroxy-5-isopropyl-phenyl)-4-(1-methyl-indol-5-yl)-5-hydroxy-[1,2,4]triazole; and
5-hydroxy-4-(5-hydroxy-4-(1-methyl-1H-indol-5-yl)-4H-1,2,4-triazol-3-yl)-2-isopropylphenyl dihydrogen phosphate;
or a tautomer or pharmaceutically acceptable salt thereof.

8. The method according to claim 7, wherein the triazolone compound is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

9. The method according to claim 7, wherein the triazolone compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

10. The method according to claim 1, wherein the non-small cell lung cancer is lung adenocarcinoma.

11. The method according to claim 10, wherein the lung adenocarcinoma is bronchioloalveolar carcinoma.

12. The method according to claim 1, wherein the non-small cell lung cancer is squamous cell lung carcinoma.

13. The method according to claim 1, wherein the non-small cell lung cancer is Stage IIIB non-small cell lung cancer.

14. The method according to claim 1, wherein the non-small cell lung cancer is Stage IV non-small cell lung cancer.

15. The method according to claim 1, wherein the triazolone compound is administered at an amount of about 200 mg/m².

16. The method according to claim 15, wherein the triazolone compound is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

17. The method according to claim 1, wherein the triazolone compound is administered in combination with one additional therapeutic agent.

18. The method of claim 17, wherein the additional therapeutic agent is docetaxel.

19. The method of claim 17, wherein the additional therapeutic agent is paclitaxel.

20. The method of claim 17, wherein the additional therapeutic agent is cisplatin.

21. The method of claim 17, wherein the method comprises administering to the mammal about 200 mg/m² of 3-(2,4-dihydroxy-5-isopropyl-phenyl)-4-(1-methyl-indol-5-yl)-5-hydroxy-[1,2,4]triazole or a tautomer or a pharmaceutically acceptable salt thereof and about 30 mg/m² of docetaxel once weekly.
